(19) United States
(12) Patent Application Publication
(10) Pub. No.: US 2006/0142223 A1
(43) Pub. Date: Jun. 29, 2006

(54) Methods for xenotopic expression of nucleus-encoded plant and protist peptides and uses thereof

(76) Inventors: Eric A. Schon, Bronx, NY (US); Joseline Ojaimi, Glen Waverley (AU)

Correspondence Address: WILMER CUTLER PICKERING HALE AND DORR LLP;COLUMBIA UNIVERSITY, 399 PARK AVENUE, NEW YORK, NY, 10020, US

(21) Appl. No.: 10/655,999

(22) Filed: Sep. 5, 2003

Related U.S. Application Data
(60) Provisional application No. 60/408,636, filed on Sep. 6, 2002.

(51) Int. Cl. A61K 48/00; C12N 9/22; C12N 15/87; C12N 15/86

(52) U.S. Cl. 514/44; 435/455; 435/456; 435/199

(57) ABSTRACT

The present invention provides a method for introducing a functional peptide encoded by a plant or protist nucleic acid sequence into a mitochondrion of a mammalian cell, and a pharmaceutical composition comprising the nucleic acid sequence. The present invention also provides a method for correcting a phenotypic deficiency in a mammal resulting from a mutation in a mitochondrial peptide. Additionally, the present invention is directed to a method for treating a mitochondrial disorder in a subject in need of treatment therefor. The present invention further provides expression vectors for use in introducing a functional peptide encoded by a plant or protist (including algal) nucleic acid sequence into a mitochondrion of a mammal, as well as mammalian cells transformed by the expression vectors. Also provided are clonal cell strains comprising the transformed mammalian cells. Finally, the present invention is directed to a method for introducing a functional peptide into a mitochondrion.

### RELATED APPLICATION

This application claims the benefit of U.S. Provisional Application No. 60/408,636, filed Sep. 6, 2002.

### STATEMENT OF GOVERNMENT INTEREST

This invention was made with government support under NIH Grant Nos. NS28828, NS39854, HD32062, and GM25661. As such, the United States government has certain rights in this invention.

### BACKGROUND OF THE INVENTION

Mitochondria are subcellular organelles found in eukaryotic cells. Under normal conditions, most of a cell's energy needs are supplied by its mitochondria. Unlike most other subcellular organelles, mitochondria are semi-independent from the nucleus, and contain their own genetic material. Mitochondrial DNA (mtDNA) was discovered in 1963 (Nass and Nass, J. Cell Biol., 19:593-629, 1963), and, by 1981, human mtDNA had been fully sequenced (Anderson et al., Nature, 290:457-65, 1981). mtDNA bears more resemblance to prokaryotic DNA than to eukaryotic DNA: (1) it is a double-stranded, circular DNA molecule; (2) the genes encoded by mtDNA do not have introns; and (3) it uses a genetic code that differs from the “universal” genetic code. Thirty-seven genes are encoded by mtDNA, thirteen of which code for peptides. In each mitochondrion, there may be 2- 10 copies of mtDNA.

There are two categories of inheritable mitochondrial disorders: those of nuclear-DNA origin and those of mtDNA origin. Since most of the proteins in mitochondria are encoded by nuclear DNA, defects in mitochondrial-protein-encoding genes in the nucleus affect mitochondrial function. For example, an A341V point mutation in NDUFVI, which is encoded by nuclear DNA, can cause patients to develop myoclonic epilepsy (Schuelke et al., Nature Genet., 21:260-61, 1999). Nevertheless, a large number of mitochondrial diseases have been linked to mtDNA abnormalities. Many of these diseases are associated with tissues that have high energy expenditures, including brain, heart, and muscle tissue.

Because mitochondria from sperm are actively degraded after fertilization, all mtDNA is inherited from the egg. Accordingly, although they can arise de novo, disorders induced by mtDNA abnormalities are more often inherited maternally. Maternally-inherited mutations in the human gene encoding ATPase 6 (MTATP6) are responsible for two related mitochondrial encephalomyopathies: NARP (neuropathy, ataxia, and retinitis pigmentosa) (Holt et al., Am. J. Hum. Genet., 46:428-33, 1990) and MILS (maternally-inherited Leigh syndrome) (Tatuch et al., Am. J. Hum. Genet., 50:852-58, 1992).

ATPase 6 is a subunit of complex V of the respiratory chain/oxidative phosphorylation system (F₀F₁-ATP synthase; E.C. 3.6.1.14), which catalyzes the synthesis of ATP from ADP and inorganic phosphate (Elston et al., 391:510-13, 1998; Noji et al., J. Biol. Chem., 276:1665-68, 2001). F₀F₁-ATP synthase is a membrane-associated polypeptide complex. In humans, the F₀F₁-ATP synthase complex comprises at least fourteen nuclear-DNA-encoded subunits (α, β, γ, δ, ε, b, c, d, e, f, g, h, IF1, and OSCP) and two mtDNA-encoded subunits (ATPase 6 {subunit a in E. coli} and ATPase 8). The F₀ portion of the complex, located in the mitochondrial inner membrane, contains a ring of c subunits surrounding a central γ subunit “stalk” that rotates within the F₁ portion of the complex—a spherical hexamer of three α-β dimers that protrudes into the matrix. ATPase 6, which is part of F₀, forms a channel through which proton flow is coupled to rotation of the c ring (Rastogi and Girvin, Nature, 402:263-68, 1999; Hutcheon et al., Proc. Natl. Acad. Sci. USA, 98:8519-24, 2001).

In both NARP and MILS, the most common mutation is a T→G point mutation at nucleotide 8993 of the ATPase 6 gene in human mtDNA (Anderson et al., Nature, 290:457-65, 1981), converting Leu-156 to Arg. In both disorders, this mutation is heteroplasmic: the patient harbors both wild-type and mutated mtDNAs, with 70-90% mutated mtDNA in NARP patients, and 90-95% in MILS patients; asymptomatic or oligosymptomatic mothers of these patients usually have less than 70% mutation in blood cells (i.e., the mutation behaves in a recessive manner). Importantly, in cells harboring high levels of the mutation, ATP synthesis is reduced by approximately 50-70% (Tatuch and Robinson, Biochem. Biophys. Res. Commun., 192:124-28, 1993; Vazquez-Memije et al., J. Inher. Metab. Dis., 19:43-50, 1996; Manfredi et al., J. Biol. Chem., 274:9386-91, 1999; Garcia et al., J. Biol. Chem., 275:11075-081, 2000).

Currently, no treatment is available for NARP, MILS, or any other mitochondrial disorders, many of which are lethal. Accordingly, there exists a need to develop therapeutic options for rescuing the deficiencies in ATP synthesis, and other deficiencies, in patients suffering from conditions associated with defects in mtDNA.

As noted above, ATPase 6 is encoded by mtDNA in humans and in almost all other organisms examined to date. Among the unicellular algae, though, only some species contain an mtDNA-encoded ATPase 6 gene, e.g., Prototheca wickerhamii, Pedinomonas minor, and the stramenopile algae, Cafeteria roenbergensis and Chrysodidymus synuroideus. In the mtDNA of other algal species, including Chlorogonium elongatum (Kroymann and Zetsche, J. Mol. Evol., 47:431-40, 1998), Chlamydomonas eugametos (Denovan-Wright et al., Plant Mol. Biol., 36:285-95, 1998), and Chlamydomonas reinhardtii (Gray and Boer, Philos. Trans. R. Soc. Lond. Biol. Sci., 319:135-47, 1988), a gene encoding ATPase 6 cannot be found. Similarly, the genes specifying COX II (including COX IIa and COX IIb) and COX III—two subunits of cytochrome c oxidase—are typically mtDNA-encoded, but are absent from the C. reinhardtii mitochondrial genome (Genbank accession number U03843). However, it has been shown that these genes are nucleus-encoded in such algal species as C. reinhardtii and Polytomella sp. (Perez-Martinez et al., J. Biol. Chem., 275:30144-152, 2000; Perez-Martinez et al., J. Biol. Chem., 276:11302-309, 2001; Perez-Martinez et al., Curr. Genet., 40:399-404, 2002; Watanabe and Ohama, J. Mol. Evol., 53:333-39, 2001).

In an attempt to elucidate new therapies for treating mitochondrial disorders, early research in yeast utilized an engineered nucleus-localized version of an mtDNA-encoded gene specifying a cytoplasmically-expressed polypeptide that could be imported into mitochondria (Law et al., FEBS Lett., 236:501-05, 1988). More recently, the present inventors showed that expression of the human mtDNA-encoded MTATP6 gene from a relocated position in the nucleus—a procedure known as “allotopic expression” (Law et al., FEBS Lett., 236:501-05, 1988; Nagley et al., Proc. Natl. Acad. Sci. USA, 85:2091-95, 1988; Claros et al., Meth. Enzymol., 264:389-403, 1996; Gray et al., Meth. Enzymol., 264:369-89, 1996; de Grey, Trends Biotechnol., 18:394-99, 2000; Zullo, S. J., Semin. Neurol., 21:327-35, 2001)—can complement a deficiency in ATP synthesis in transmitochondrial cells harboring the T8993G mutation associated with NARP and MILS (Manfredi et al., Nature Genet., 25:394-99, 2002). In particular, in their earlier studies, the inventors successfully rescued ATP synthesis in mitochondria of mammalian cells by allotopically expressing wild-type ATPase 6 protein from nucleus-transfected constructs encoding an amino-terminal mitochondrial-targeting signal appended to a recoded ATPase 6 gene (rendered compatible with the universal genetic code) that also contained a carboxy-terminal FLAG epitope tag. After transfection of human cells, the precursor polypeptide was expressed, imported into and processed within mitochondria, and incorporated into complex V (Manfredi et al., Nature Genet., 25:394-99, 2002). However, prior to the present invention, it was not known that the ATPase 6 gene from Chlamydomonas reinhardtii—which is nucleus-encoded (Funes et al., J. Biol. Chem., 277:6051-58, 2002)—can be expressed in human cells, and can rescue ATP-synthesis defects in human cells harboring an mtDNA mutation.

### SUMMARY OF THE INVENTION

Unlike most organisms, the mitochondrial genome of Chlamydomonas reinhardtii, a unicellular green alga, does not contain a gene encoding subunit 6 of F₀F₁-ATP synthase (ATPase 6). The inventors hypothesized that the C. reinhardtii ATPase 6 is nucleus-encoded, and identified cDNAs and a single-copy nuclear gene (CrATP6) specifying this subunit. The CrATP6 gene consists of eight exons, of which the first four appear to encode a putative mitochondrial-targeting signal (MTS). Although the algal and human ATPase 6 polypeptides are highly diverged, their secondary structures are remarkably similar.

The inventors provide evidence herein that, when CrATP6 is expressed in human cells, a significant amount of the precursor polypeptide is targeted to mammalian mitochondria, the MTS is cleaved within the organelle, and the mature polypeptide is assembled into human complex V. Furthermore, the inventors demonstrate that, in spite of the evolutionary distance between algae and mammals, C. reinhardtii ATPase 6 can function in human cells. Specifically, the inventors show that deficiencies in both cell viability and ATP synthesis in transmitochondrial cell lines harboring a pathogenic mutation in the human mtDNA-encoded ATP6 gene may be overcome by expression of CrATP6 in these cells. It is believed that the ability to express a nucleus-encoded version of a mammalian mtDNA-encoded protein may provide a novel mechanism for importing other highly hydrophobic proteins into mitochondria, and may also serve as the basis for a gene-therapy approach to treat human mitochondrial diseases.

Accordingly, the present invention provides a method for introducing a functional peptide encoded by a plant or protist nucleic acid sequence into a mitochondrion of a mammalian cell, by: (a) preparing a nucleic-acid construct comprising a plant or protist nucleic acid sequence encoding the peptide and, optionally, a plant or protist nucleic acid sequence encoding a mitochondrial-targeting signal; (b) introducing the nucleic-acid construct into a mammalian cell to produce a transformed cell; and (c) expressing the nucleic-acid construct from the nucleus of the transformed cell.

The present invention also provides a method for correcting a phenotypic deficiency in a mammal that results from a mutation in a mitochondrial peptide, by: (a) establishing the identity of the mitochondrial peptide having the mutation; (b) preparing a nucleic-acid construct comprising a plant or protist nucleic acid sequence encoding the peptide and, optionally, a plant or protist nucleic acid sequence encoding a mitochondrial-targeting signal, wherein the plant or protist nucleic acid sequence encoding the peptide encodes a functional peptide; (c) introducing the nucleic-acid construct into a mammalian cell to produce a transformed cell; and (d) expressing the nucleic-acid construct from the nucleus of the transformed cell.

Additionally, the present invention provides a method for treating a mitochondrial disorder in a subject in need of treatment therefore, by administering to the subject a functional plant or protist peptide in an amount effective to treat the mitochondrial disorder.

The present invention further provides an expression vector for use in introducing a functional peptide encoded by an algal nucleic acid sequence into a mitochondrion of a mammal, comprising a nucleic acid sequence encoding Chlamydomonas reinhardtii ATPase 6 subunit of F₀F₁-ATP synthase or the mitochondrial-targeting signal thereof. Also provided is a mammalian cell transformed by the expression vector.

The present invention is also directed to a mammalian cell transformed by an expression vector for use in introducing a functional peptide encoded by a plant or protist nucleic acid sequence into a mitochondrion, wherein the expression vector comprises a plant or protist nucleic acid sequence encoding the peptide and, optionally, a plant or protist nucleic acid sequence encoding a mitochondrial-targeting signal. Also provided is a clonal cell strain comprising the transformed mammalian cell.

The present invention further provides a pharmaceutical composition, comprising: (a) a plant or protist nucleic acid sequence encoding a peptide for introduction into a mitochondrion; (b) optionally, a plant or protist nucleic acid sequence encoding a mitochondrial-targeting signal; and (c) a pharmaceutically-acceptable carrier.

Finally, the present invention is directed to a method for introducing a functional peptide into a mitochondrion, by: (a) preparing a nucleic-acid construct comprising a nucleic acid sequence encoding the peptide and a nucleic acid sequence encoding the mitochondrial-targeting sequence of Chlamydomonas reinhardtii ATPase 6 subunit of F₀F₁-ATP synthase; (b) introducing the nucleic-acid construct into a eukaryotic cell to produce a transformed cell, wherein the eukaryotic cell is derived from an animal, a plant, a fungus, or a protozoan; and (c) expressing the nucleic-acid construct from the nucleus of the transformed cell.

Additional aspects of the present invention will be apparent in view of the description which follows.

### BRIEF DESCRIPTION OF THE FIGS.

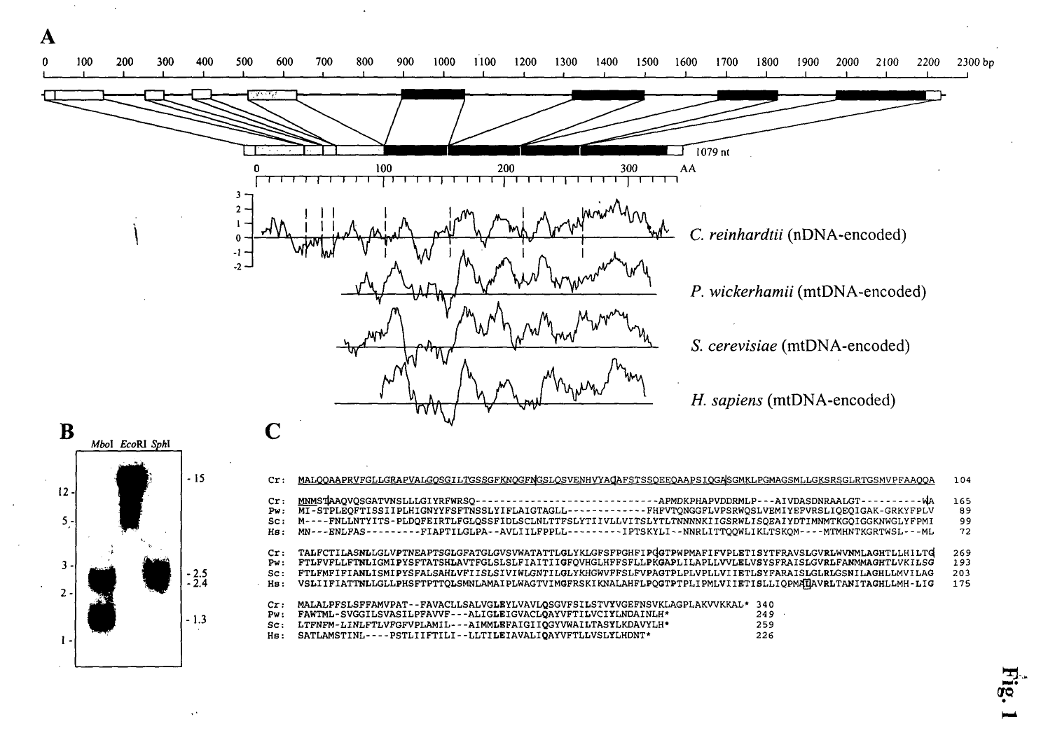

FIG. 1 depicts the characterization of the C. reinhardtii ATP6 (CrA TP6) gene and the protein encoded thereby (ATPase 6). A: The gene (upper map) and processed mRNA (lower map) contain eight exons (boxes), of which the first four (gray shading) encode the mitochondrial-targeting signal (MTS) (Funes et al., J. Biol. Chem., 277:6051-58, 2002) and the last four (black shading) encode the mature protein; the 5′ and 3′ UTRs are unshaded. Below the maps are Kyte-Doolittle hydropathy plots (hydropathy scale at left) for ATPase 6, from the indicated organisms. Dashed lines denote exon-intron boundaries in CrATP6. B: Southern-blot hybridization of genomic C. reinhardtii DNA digested with the indicated restriction enzymes and probed with the coding region of CrATP6 cDNA. Markers, in kilobases (kb), are at the left; the approximate sizes of the hybridizing bands, in kb, are at the right. C: Alignments of ATPase 6 polypeptides from C. reinhardtii (SEQ ID NO: 1), P. wickerhamii (SEQ ID NO:2), S. cerevisiae (SEQ ID NO:3), and H. sapiens (SEQ ID NO:4). Amino acid numbering is at the right. Exon-intron boundaries for CrATP6 are indicated by the vertical lines, and the MTS is underlined. The MTS of CrATP6 is located at 1-107aa (Funes et al., J. Biol. Chem., 277:6051-58, 2002), with cleavage between Met-107 and Ser-108. Leu-156 in human ATPase 6, which is mutated in NARP/MILS, is boxed. Residues conserved among all four species are in bold. nt=nucleotide; aa=amino acid

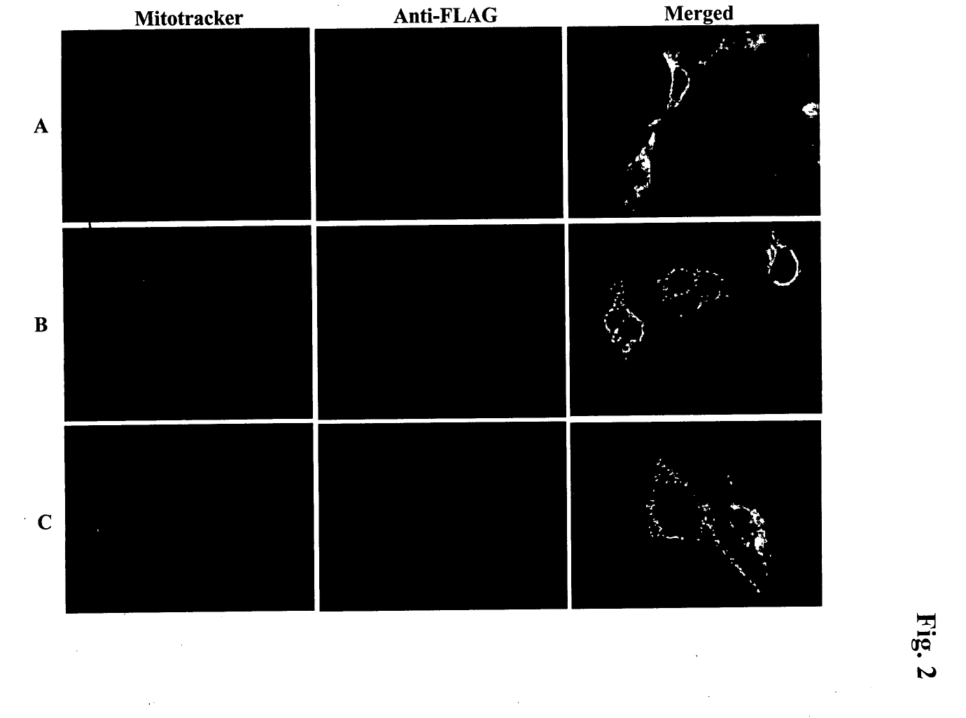

FIG. 2 illustrates the targeting of C. reinhardtii ATPase 6 to mammalian mitochondria. pCrA6F was expressed transiently in human 293T HEK (A) and monkey COS7 (B) SV40-transformed kidney cells, and stably in human cybrid line JCP261 harboring 100% mutated (i.e., 8993G) mtDNAs from an MILS patient (C). Immunohistochemistry using an anti-FLAG antibody was carried out to detect the FLAG epitope tag. Note the co-localization of the anti-FLAG signal (green fluorescence) with that of Mitotracker Red (red fluorescence) in the merged panels at the right.

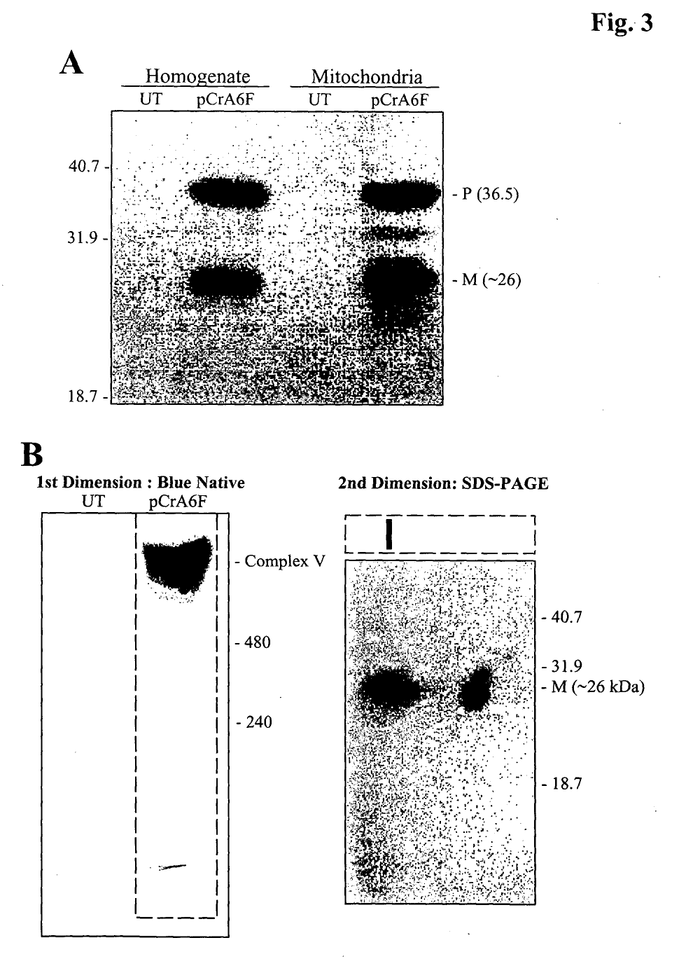

FIG. 3 sets forth Western-blot analyses. (A) Western blot of proteins isolated from human 293T HEK cells transfected transiently with pCrA6F, or from untransfected cells (IT), using anti-FLAG antibodies. Sizes of markers, in kilodaltons (kDa), are at left. The predicted sizes of the precursor (P) and mature (M) CrA6F polypeptides, in kDa, are at right. (B) Proteins from human 293T HEK cells were electrophoresed through 5-18% continuous-gradient blue-native gels run in parallel. One gel was transferred onto PVDF membranes, and was probed with anti-FLAG antibodies. This probing revealed a signal migrating at ˜600 kDa, determined previously by the inventors to be complex V (Manfredi et al., Nature Genet., 25:394-99, 2002) (left panel). A lane from the parallel gel was excised (dashed box); run in the second dimension, under denaturing conditions, through a 15% SDS-PAGE gel, with the top of the gel oriented as shown (right panel); and probed with anti-FLAG antibodies. Markers, in kDa, are at the right in both gels.

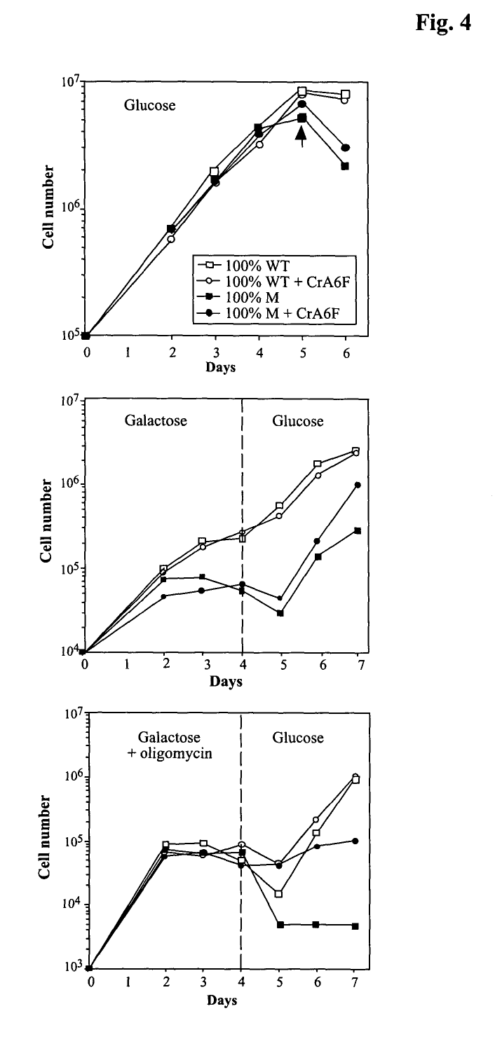

FIG. 4 depicts growth curves for cybrids grown in glucose only (upper panel), galactose followed by glucose (middle panel), or galactose+oligomycin followed by glucose (lower panel). Shown is an example of a set of growth curves of cybrids containing 100% wild-type (WT) (open symbol) and 100% mutant (M) (filled symbol) mtDNAs, either untransfected (square) or transfected with pCrA6F (circle), and grown in the indicated media. Arrow denotes confluency.

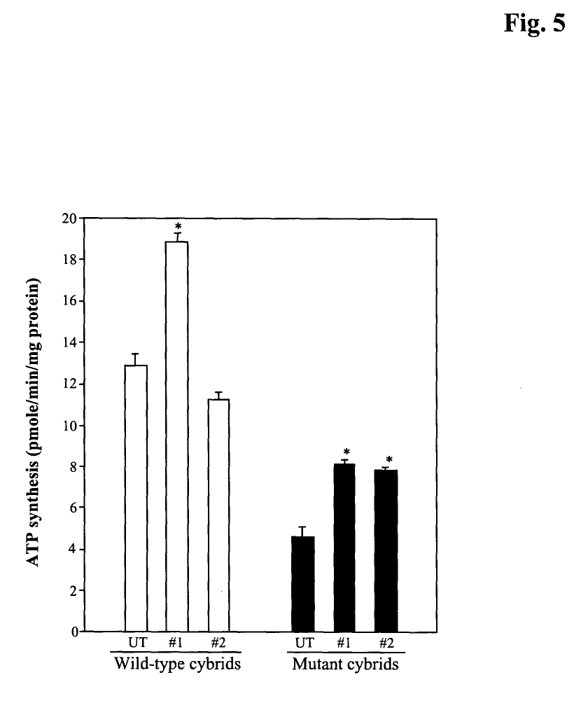

FIG. 5 illustrates ATP synthesis. Measurement of oligomycin-sensitive (i.e., mitochondrial) ATP synthesis using malate/pyruvate as the substrate, in wild-type and mutant cybrids transfected with pCrA6F in two independent transfections (#1 and #2), is compared with values in the respective untransfected (UT) cells. Measurements were performed in triplicate. Asterisks denote values that were statistically significant (p<0.05, in a paired Student's t-test), as compared with those from the respective untransfected cells.

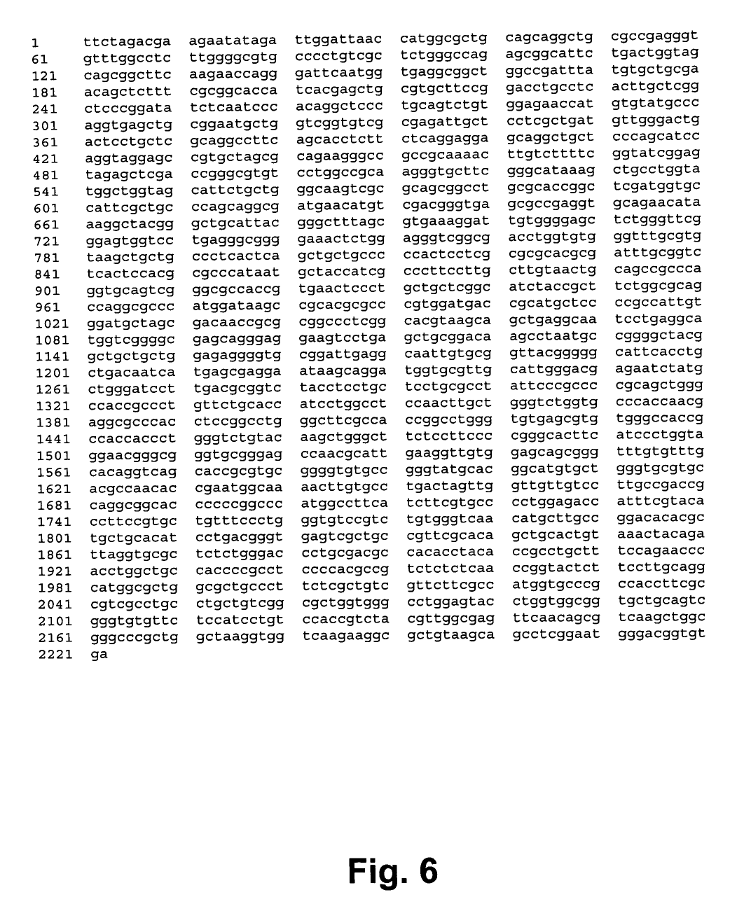

FIG. 6 sets forth the genomic nucleotide sequence of C. reinhardtii ATPase 6 (SEQ ID NO:6). Cleavage of the mitochondrial-targeting signal (Funes et al., J. Biol. Chem., 277:6051-58, 2002) occurs after 629nt.

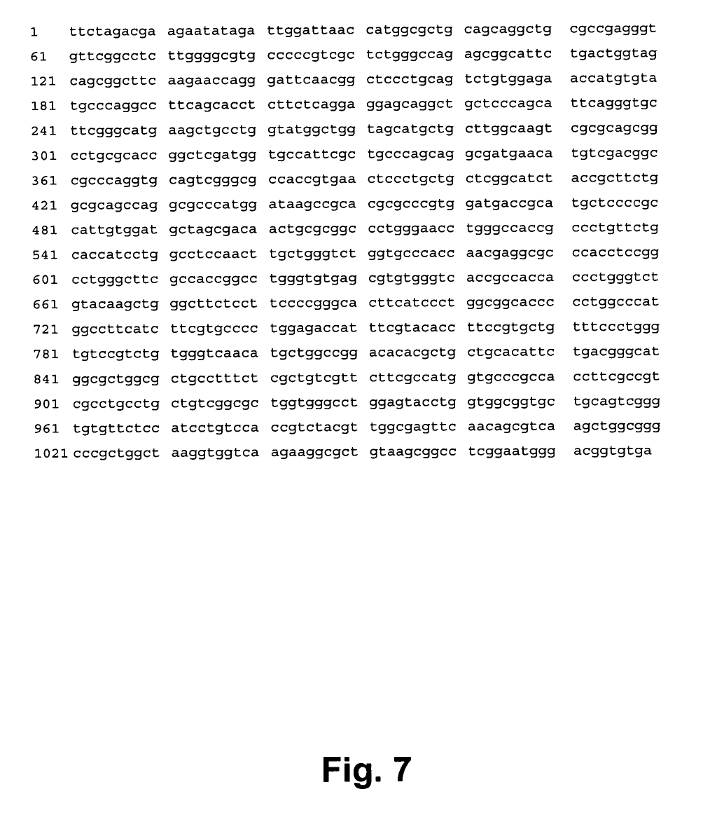

FIG. 7 sets forth the cDNA nucleotide sequence of C. reinhardtii ATPase 6 (SEQ ID NO:7). Cleavage of the mitochondrial-targeting signal (Funes et al., supra) occurs after 352nt.

### DETAILED DESCRIPTION OF THE INVENTION

Because the mitochondrial F₀F₁-ATP synthase in C. reinhardtii is sensitive to oligomycin (Nurani and Franzen, Plant Mol. Biol., 31:1105-16, 1996), and oligomycin sensitivity is conferred by ATPase 6 (Breen et al., J. Biol. Chem., 261:11680-685, 1986; John and Nagley, FEBS Lett., 207:79-83, 1986), the inventors reasoned that a gene specifying this subunit is encoded in the C. reinhardtii nuclear genome, rather than the C. reinhardtii mitochondrial DNA (mtDNA). In support of this view, it had already been shown that the genes specifying COX II (including COX IIa and COX IIb) and COX III—two subunits of cytochrome c oxidase that are typically mtDNA-encoded, but which are also absent from the C. reinhardtii mitochondrial genome (Genbank accession number U03843)—are nuclear-encoded instead (Perez-Martinez et al., J. Biol. Chem., 275:30144-152, 2000; Perez-Martinez et al., J. Biol. Chem., 276:11302-309, 2001; Perez-Martinez et al., Curr. Genet., 40:399-404, 2002; Watanabe and Ohama, J. Mol. Evol., 53:333-39, 2001).

The inventors recently showed that expression of the human mtDNA gene encoding ATPase 6 (MTA TP6) from a relocated position in the nucleus—a procedure referred to as “allotopic expression” (Law et al., FEBS Lett., 236:501-05, 1988; Nagley et al., Proc. Natl. Acad. Sci. USA, 85:2091-95, 1988; Claros et al., Meth. Enzymol., 264:389-403, 1996; Gray et al., Meth. Enzymol., 264:369-89, 1996; de Grey, Trends Biotechnol., 18:394-99, 2000; Zullo, S. J., Semin. Neurol., 21:327-35, 2001)—can complement a deficiency in ATP synthesis in transmitochondrial cells harboring the T8993G mutation associated with NARP and MILS (Manfredi et al., Nature Genet., 25:394-99, 2002). Herein, the inventors demonstrate that the nucleus-encoded ATP6 gene from Chlamydomonas reinhardtii (Funes et al., J. Biol. Chem., 277:6051-58, 2002) (CrATP6) can be expressed in human cells, and can also rescue ATP-synthesis defects in human cells harboring this mtDNA mutation.

Accordingly, the present invention provides a method for introducing a peptide into a mitochondrion. Unless otherwise indicated, “peptide” shall include a protein, protein domain, polypeptide, peptide, or amino acid sequence, including any post-translational modification(s). One of skill in the art, upon reading the instant specification, will appreciate that these terms also include structural analogs and derivatives, e.g., peptidomimetics, peptides having conservative amino acid insertions, deletions, or substitutions, and the like. A mitochondrion is a membrane-bound organelle that compartmentalizes energy production within a eukaryotic cell.

In a particular aspect, the present invention provides a method for introducing a functional peptide encoded by a plant or protist nucleic acid sequence into a mitochondrion of a mammalian cell. As used herein, the term “functional peptide” refers to a peptide that demonstrates biological activity or function in a manner for which it was intended, and does not display a modification in its activity or functional properties as compared with the wild-type, or non-mutant, peptide. For example, where the peptide is an enzyme, or part of an enzyme complex, the peptide is functional if it demonstrates enzymatic activity (e.g., catalytic activity). The function of a peptide may be determined by standard assays that are well-known in the art, including those described herein.

As further used herein, the term “plant or protist nucleic acid sequence” describes a nucleic acid sequence, or sequence of nucleotides (including DNA and RNA), that originates within a plant or protist. A plant is a member of the kingdom plantae. Members of the plantae kingdom are multicellular, eukaryotic organisms that usually conduct photosynthesis. Examples of plants for use in the present invention include, without limitation, angiosperms (Adams et al., Plant Cell, 14:931-943, 2002), rice (Kubo et al., Mol. Gen. Genet., 263:733-39, 2000), soybean and other legumes (Covello et al., EMBO J., 11:3815-20, 1992; Daley et al., Proc. Natl. Acad. Sci. USA, 99:10510-515, 2002; Daley et al., Plant J., 30:11-21, 2002), and some types of algae. A protist is a member of the kingdom protista. Members of the protista kingdom are single-celled or multicellular eukaryotic organisms. Protists are more complex than bacteria—the prokaryotic members of the kingdom monera. Examples of protists for use in the present invention include, without limitation, apicomplexans (e.g., Plasmodium falciparum and Plasmodium reinchenowi), ciliates (e.g., Paramecium aurelia and Tetrahymena pyriformia), paramecia, protozoa, Trypanosoma brucei (Tan et al., Mol. Cell Biol., 22:3707-17, 2002), and some types of algae. In one embodiment of the present invention, the plant or protist nucleic acid sequence is an algal nucleic acid sequence (i.e., the nucleic acid sequence originates within an alga). Algae are phototrophic, eukaryotic microorganisms that may be classed as plants or protists. They comprise a large group of diverse unicellular and multicellular aquatic organisms, including plants, and grow in both fresh water and sea water.

The plant or protist (including algal) nucleic acid sequences of the present invention may be nuclear or non-nuclear (e.g., mtDNA). As used herein, the term “nuclear” describes a nucleic acid sequence, or sequence of nucleotides (including DNA and RNA), that originates within the nucleus of a cell, and includes chromosomal DNA. Contrastingly, the term “non-nuclear” describes a nucleic acid sequence, or sequence of nucleotides (including DNA and RNA), that originates outside of the nucleus of a cell, and includes extrachromosomal DNA. For example, a non-nuclear nucleic acid sequence could originate in an organelle (e.g., a chloroplast or a mitochondrion) or in the cytoplasm of the cell.

In the method of the present invention, the peptide encoded by a nuclear or non-nuclear nucleic acid sequence may be any plant or protist peptide (i.e., any peptide that originates within a plant or protist), especially any plant or protist peptide that is encoded by nuclear DNA in the plant or protist, but is encoded by mtDNA in mammals. Examples of plant or protist peptides for use in the present invention include, without limitation, rps8 and rps13 from angiosperms (Adams et al., Plant Cell, 14:931-943, 2002), rps10 from rice (Kubo et al., Mol. Gen. Genet., 263:733-39, 2000), and COX II from soybean and other legumes (Covello et al., EMBO J., 11:3815-20, 1992; Daley et al., Proc. Natl. Acad. Sci. USA, 99:10510-515, 2002; Daley et al., Plant J., 30:11-21, 2002).

In one embodiment of the present invention, the peptide is encoded by algal mitochondrial DNA (mtDNA). Examples of peptides encoded by algal mtDNA include, without limitation, ATPase 8 subunit of F₀F₁-ATP synthase and ATPase 6 of Prototheca wickerhamii, Pedinomonas minor, Cafeteria roenbergensis, and Chrysodidymus synuroideus. In a preferred embodiment of the present invention, however, the peptide is encoded by algal nuclear DNA. Examples of peptides encoded by algal nuclear DNA include, without limitation, ATPase 6 subunit of F₀F₁-ATP synthase (ATPase 6) (e.g., from Chlamydomonas reinhardtii); ATPase 8 subunit of F₀F₁-ATP synthase (ATPase 8) and ATPase 9 subunit of F₀F₁-ATP synthase (ATPase 9); a cytochrome c oxidase subunit, such as COX II (including COX IIa and COX IIb) or COX III—two subunits of cytochrome c oxidase (e.g., from C. reinhardtii or Polytomella sp.); ATPase 6 from Chlorogonium elongatum and Chlamydomonas eugametos; and other peptides that are encoded by nuclear DNA in algae, but are encoded by mtDNA in mammals. Preferably, the peptide is C. reinhardtii ATPase 6 subunit of F₀F₁-ATP synthase.

The method of the present invention comprises the steps of: (a) preparing a nucleic-acid construct comprising a plant or protist (including algal) nucleic acid sequence encoding a peptide for introduction into a mitochondrion of a mammalian cell and, optionally, a plant or protist (including algal) nucleic acid sequence encoding a mitochondrial-targeting signal; (b) introducing the nucleic-acid construct into a mammalian cell to produce a transformed cell; and (c) expressing the nucleic-acid construct from the nucleus of the transformed cell. The functional peptide that is expressed may then be targeted to, and introduced into, the mitochondrion of the mammalian cell under direction of the mitochondrial-targeting signal. Where the nucleic acid sequence is non-nuclear, it may be necessary to render the nucleic acid sequence compatible with the universal genetic code, prior to including it in the nucleic-acid construct, in accordance with methods described below.

In the method of the present invention, the plant or protist (including algal) nucleic acid sequences may be DNA or RNA (e.g., nuclear DNA or RNA, or non-nuclear (including mitochondrial) DNA or RNA), including synthetic forms and mixed polymers, and sense and antisense strands. Nucleic acid sequences for use in the method of the present invention may be isolated from cell cultures using known methods. Additionally, the nucleic acid sequences may be prepared by a variety of techniques known to those skilled in the art, including, without limitation: restriction enzyme digestion of nucleic acid; and automated synthesis of oligonucleotides, using commercially-available oligonucleotide synthesizers such as the Applied Biosystems Model 392 DNA/RNA synthesizer.

A “mitochondrial-targeting signal”, as used herein, is a peptide sequence, encoded by a nucleic acid sequence, that directs a peptide to its target, the mitochondrion. Many genes originally present in mitochondria and chloroplasts have been relocated, through time, to nuclear genomes. The products of their expression are targeted back to the appropriate organelles under the direction of organelle-targeting signals or transit peptides. Accordingly, the mitochondrial-targeting signal of the present invention may be a peptide sequence that occurs in nature, which is added to a nuclear-DNA-encoded peptide that is generally transported to a mitochondrion. Additionally, the mitochondrial-targeting signal of the present invention may be an artificial, or synthetic, peptide sequence which may correspond to a naturally-occurring transit sequence. The plant or protist (including algal) nucleic acid sequence encoding the peptide of the present invention may be derived from the same species as, or a different species from, that from which the plant or protist nucleic acid encoding the mitochondrial-targeting signal of the present invention is derived.

Where a plant or protist (including algal) nucleic acid sequence encoding a peptide is nuclear DNA, it may be expected that the peptide produced therefrom will be a precursor polypeptide that includes, or is contiguous with, a mitochondrial-targeting signal. For example, the C. reinhardtii A TP6 (CrA TP6) gene encodes a precursor polypeptide encompassing an ATPase 6 peptide along with its mitochondrial-targeting signal. However, where the plant or protist (including algal) nucleic acid sequence encoding a peptide is non-nuclear (e.g., mtDNA), the peptide produced therefrom may not include a mitochondrial-targeting signal. Accordingly, the method of the present invention provides the option of including in the nucleic-acid construct a plant or protist (including algal) nucleic acid sequence encoding a mitochondrial-targeting signal. Examples of mitochondrial-targeting signals that may be useful in the method of the present invention include, without limitation, the 32-amino-acid chloroplast transit sequence of the ribulose bisphosphatase carboxylase/oxygenase activase preprotein from Chlamydomonas reinhardtii; the N-terminal amphipathic alpha-helices of cellular proteins; proteins that comprise the tetratricopeptide repeat (TPR), having four copies of the 34-amino-acid TPR motif; the primary sequence of the amino terminus of the Arabidopsis biotin synthase; the mitochondrial-targeting signal of C. reinhardtii ATPase 6 subunit of F₀F₁-ATP synthase (1-107aa of SEQ ID NO:1); and other N-terminal hydrophilic or hydrophobic signal peptides. Preferably, the mitochondrial-targeting signal is the mitochondrial-targeting signal of C. reinhardtii ATPase 6 subunit of F₀F₁-ATP synthase.

In accordance with the method of the present invention, a nucleic-acid construct may be prepared by methods known in the art, including those described below. Vectors, promoters, and ribosomal entry sites, including those disclosed herein, may be used in conjunction with standard techniques to prepare the nucleic-acid construct of the present invention. Vectors that may be useful in the present invention include, without limitation, pCDNA3, bicistronic vectors (e.g., pEF-BOS-IRES), plasmid vectors (e.g., plasmid pcDNA3/5a-a vector, referred to herein as “pCrA6F”), and adeno-associated virus (AAV) vectors (e.g., pTR-UF5, pTR-UF11, and PTR-UF12).

In the method of the present invention, the nucleic-acid construct may be labeled with a detectable marker, for facilitating detection of the peptide encoded within the nucleic-acid construct. Labeling may be accomplished using one of a variety of labeling techniques, including peroxidase, chemiluminescent labels known in the art, and radioactive labels known in the art. Additional detectable markers which may be useful in the method of the present invention include, without limitation, nonradioactive or fluorescent markers, such as biotin, fluorescein (FITC), acridine, cholesterol, and carboxy-X-rhodamine, which can be detected using fluorescence and other imaging techniques readily known in the art. Alternatively, the detectable marker may be a radioactive marker, including, for example, a radioisotope. The radioisotope may be any isotope that emits detectable radiation, such as ³⁵S, ³²P, or ³H. Radioactivity emitted by the radioisotope can be detected by techniques well known in the art. For example, gamma emission from the radioisotope may be detected using gamma imaging techniques, particularly scintigraphic imaging.

In accordance with the method of the present invention, the detectable marker may be encoded by a nucleic acid sequence that is incorporated within the nucleic-acid construct, resulting in expression of the detectable marker when the peptide of the present invention is expressed. Accordingly, in one embodiment of the present invention, the nucleic-acid construct further comprises a nucleic acid sequence encoding a detectable marker (e.g., an immunohistochemical marker). In a preferred embodiment of the present invention, the detectable marker is a FLAG epitope. This marker may be detected using anti-FLAG antibodies and Western-blot analysis.

In the method of the present invention, the nucleic-acid construct is introduced into a mammalian cell, in a manner permitting expression of the peptide encoded by the plant or protist (including algal) nucleic acid sequence within the construct, thereby producing a transformed cell. The mammalian cell may be derived from any mammal (e.g., humans, domestic animals, and commercial animals), and may be any type of cell, including a bone-marrow cell, a germ-line cell, a post-mitotic cell (e.g., a cell of the central nervous system), a progenitor cell, and a stem cell. In one embodiment of the present invention, the cell is a human cell, which may include a cell from a human cell line (e.g., a human 293T HEK cell). In a preferred embodiment, the mammalian cell is a human cell and the peptide is C. reinhardtii ATPase 6 subunit of F₀F₁-ATP synthase.

The nucleic-acid construct of the present invention may be introduced into a mammalian cell by standard methods of transfection or transformation known in the art. Examples of methods by which the construct may be introduced into the cell include, without limitation, electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus.

It is within the confines of the present invention that the nucleic-acid construct may be introduced into a mammalian cell in vitro, using conventional procedures, to achieve expression of the plant or protist (including algal) peptide in the cell. This procedure may be replicated multiple times, to obtain expression of the peptide in multiple mammalian cells. Mammalian cells expressing the peptide then may be introduced into a mammal, such that the functional peptide is expressed within mitochondria in vivo. In such an ex vivo (outside of the mammal) gene therapy approach, the mammalian cells are preferably removed from the mammal, subjected to DNA techniques to incorporate the nucleic-acid construct, and then reintroduced into the mammal. However, the mammalian cells also may be derived from another mammalian organism, either of the same, or a different, species.

In the method of the present invention, the nucleic-acid construct is expressed from the nucleus of the mammalian cell into which it has been introduced. As used herein, the term “expressed from the nucleus” means that the transcription machinery of the nucleus, rather than the transcription machinery of a mitochondrion or other organelle, is used to generate an mRNA transcript of the peptide encoded by the plant or protist (including algal) nucleic acid sequence. Thereafter, the mRNA transcript is shuttled to the cytoplasm of the mammalian cell, wherein the transcript is translated into a functional peptide. The transcription-translation mechanisms of mitochondria or other organelles are not involved.

Expression of a plant or protist (including algal) peptide from the nucleus of a mammalian cell is an example of “xenotopic” expression or “trans-kingdom allotopic” expression. As disclosed herein, where the peptide is encoded by mtDNA, and it is necessary to include in the nucleic-acid construct a plant or protist (including algal) nucleic acid sequence encoding a mitochondrial-targeting signal, the nucleic acid sequence encoding the plant or protist mitochondrial-targeting signal is transcribed and translated along with the plant or protist nucleic acid sequence encoding the peptide, such that the expressed peptide bears the mitochondrial-targeting signal. It is this signal that then directs the expressed peptide in the cytoplasm of the mammalian cell to its targeted organelle, the mitochondrion.

Expression of the plant or protist (including algal) peptide may be detected in the mammalian cell by detection methods readily determined from the known art, including, without limitation, immunological techniques (e.g., binding studies and Western blotting), hybridization analysis (e.g., using nucleic acid probes), fluorescence imaging techniques, and/or radiation detection. Similarly, the mammalian cell may be assayed for peptide function, using standard protein assays known in the art or disclosed herein.

As described above, the method of the present invention may be used to introduce a peptide into a mitochondrion in vitro, or in vivo in a mammal, by introducing the nucleic-acid construct of the present invention into a sufficient number of cells of the mammal (either in situ or ex vivo), in a manner permitting expression of the peptide encoded by the plant or protist (including algal) nucleic acid sequence (and, optionally, the mitochondrial-targeting signal) contained within the construct. In view of the foregoing, the transformed mammalian cell of the present invention may be in, or introduced into, a mammal. The mammal may be any mammalian animal (e.g., a human, domestic animal, or commercial animal), but is preferably a human.

Where the mammalian cell is already in a human, the mitochondrion may be contained within any cell of the human, including bone-marrow cells, germ-line cells, post-mitotic cells (e.g., cells of the central nervous system), progenitor cells, and stem cells. Where ex vivo gene therapy techniques are used, the mitochondrion may be initially contained within a mammalian cell (including a bone-marrow, germ-line, post-mitotic, progenitor, or stem cell) outside of the human, wherein the cell is preferably from the human species, and, more preferably, from the human subject. The mammalian cell containing the functional peptide within the mitochondrion target then may be introduced into the human to permit in vivo proliferation of cells containing the functional peptide within their mitochondria. The mammalian cell may be introduced into the human by standard techniques known in the art, including injection and transfusion.

In accordance with the present invention, the use of xenotopic expression to introduce a functional peptide encoded by a plant or protist (including algal) nucleic acid sequence into a mitochondrion of a mammalian cell may be utilized to rescue a mitochondrial disorder, such as a deficiency in ATP synthesis resulting from a defect in an mtDNA gene (e.g., MTATP6). Without being bound by theory, it is believed that, by providing a method for introducing functional plant and protist (including algal) peptides into mammalian mitochondria, the xenotopic-expression method of the present invention will be useful for the treatment of conditions associated with defects in mtDNA that result in defective peptides within the mitochondria. Thus, the method of the present invention may be particularly useful for treating mitochondrial disorders. Accordingly, the present invention provides a method for treating a mitochondrial disorder in a human in need of treatment, comprising introducing to the human the nucleic-acid construct of the present invention.

As used herein, a “mitochondrial disorder” is a condition, disease, or disorder characterized by a defect in activity or function of mitochondria, particularly a defect in mitochondrial activity or function that results from, or is associated with, a mutation in mtDNA. Examples of mitochondrial disorders include, without limitation, aging; aminoglycoside-induced deafness; cardiomyopathy; CPEO (chronic progressive external ophthalmoplegia); encephalomyopathy; FBSN (familial bilateral striatal necrosis); KS (Kearns-Sayre) syndrome; LHON (Leber's hereditary optic neuropathy); MELAS (mitochondrial myopathy, encephalopathy, lactic acidosis, and stroke-like episodes); MERRF (myoclonic epilepsy with stroke-like episodes); MILS (maternally-inherited Leigh syndrome); mitochondrial myopathy; NARP (neuropathy, ataxia, and retinitis pigmentosa); PEO; and SNE (subacute necrotizing encephalopathy).

In one embodiment of the present invention, the mitochondrial disorder is associated with a mutation (e.g., a point mutation) in mtDNA. A “mutation”, as used herein, is a permanent, transmissable change in genetic material. In another embodiment of the present invention, the mitochondrial disorder in the human is FBSN, MILS, or NARP, and the mammalian cell of the present invention (either in, or introduced into, the human) is transformed with a nucleic-acid construct comprising an algal nucleic acid sequence that encodes functional C. reinhardtii ATPase 6 subunit of F₀F₁-ATP synthase. A functional peptide may be a wild-type peptide. As used herein, the term “wild-type” refers to the characteristic genotype (or phenotype) for a particular gene (or its gene product), as found most frequently in its natural source (e.g., in a natural population). A wild-type animal, for example, expresses functional ATPase 6 subunit of F₀F₁-ATP synthase.

The present invention is also directed to a method for correcting a phenotypic deficiency in a mammal that results from a mutation in a mitochondrial peptide of the mammal. As used herein, the term “phenotypic deficiency” refers to a defect in a mammal that manifests, at a cellular level, as subnormal activity or function of one or more peptides in the mammal, and can result in a condition, disease, or disorder in the mammal. As further used herein, the term “mitochondrial peptide” refers to a peptide that originates within, or is transferred to, the mitochondrion for use within, or by, that organelle. In the method of the present invention, the mutation in the mitochondrial peptide, and the resulting phenotypic deficiency in the mammal, may be caused by a mutation in the mammal's nuclear DNA or non-nuclear DNA (e.g., mtDNA). By way of example, where a human has a mutation in the nuclear-DNA-encoded MTATP6 gene, ATP synthesis at the cellular level may be below the level normally expected in a healthy human, resulting in a mitochondrial disorder, such as FBSN, MILS, or NARP.

As further used herein, the term “correcting a phenotypic deficiency” means rescuing or minimizing the phenotypic deficiency by restoring, or partially restoring, at the cellular level, the activity or function of the defective peptide, thereby treating the condition, disease, or disorder in the mammal. Where, for example, a human suffers from a mitochondrial disorder such as FBSN, MILS, or NARP, as a result of a mutation in the MTATP6 gene, the phenotypic deficiency of defective ATP synthesis may be corrected by restoring, or partially restoring, activity or function of ATPase 6, thereby treating the mitochondrial disorder.

Accordingly, the method of the present invention comprises the steps of: (a) establishing the identity of the mitochondrial peptide having the mutation of interest; (b) preparing a nucleic-acid construct comprising a plant or protist (including algal) nucleic acid sequence encoding the peptide and, optionally, a plant or protist (including algal) nucleic acid sequence encoding a mitochondrial-targeting signal, wherein the plant or protist nucleic acid sequence encoding the peptide encodes a functional (e.g., wild-type) peptide; (c) introducing the nucleic-acid construct into a mammalian cell to produce a transformed cell; and (d) expressing the nucleic-acid construct from the nucleus of the transformed cell. The functional peptide that is expressed in the cytosol of the cell may then be targeted to, and introduced into, the mitochondrion under direction of the mitochondrial-targeting signal.

If the identity of the peptide having the mutation of interest is not already known, or has not already been established, it is possible to establish the identity of the peptide by using standard techniques known in the art for isolating and analyzing mtDNA to determine genetic defects, or for isolating and analyzing protein to determine phenotypic defects. The plant or protist (including algal) nucleic acid sequences of the present invention may be nuclear or non-nuclear (e.g., mtDNA). Examples of plant or protist peptides for use in the present invention include, without limitation, rps8 and rps13 from angiosperms (Adams et al., Plant Cell, 14:931-943, 2002), rps10 from rice (Kubo et al., Mol. Gen. Genet., 263:733-39, 2000), and COX II from soybean and other legumes (Covello et al., EMBO J., 11:3815-20, 1992; Daley et al., Proc. Natl. Acad. Sci. USA, 99:10510-515, 2002; Daley et al., Plant J., 30:11-21, 2002).

Examples of peptides encoded by algal mtDNA include, without limitation, ATPase 8 subunit of F₀F₁-ATP synthase and ATPase 6 of Prototheca wickerhamii, Pedinomonas minor, Cafeteria roenbergensis, and Chrysodidymus synuroideus. Where the nucleic acid sequence is non-nuclear, it may be necessary to render the nucleic acid sequence compatible with the universal genetic code, prior to including it in the nucleic-acid construct, in accordance with methods described below. In a preferred embodiment of the present invention, the peptide is encoded by algal nuclear DNA. Examples of peptides encoded by algal nuclear DNA include, without limitation, ATPase 6 subunit of F₀F₁-ATP synthase (e.g., from C. reinhardtii); ATPase 8 subunit of F₀F₁-ATP synthase and ATPase 9 subunit of F₀F₁-ATP synthase; a cytochrome c oxidase subunit, such as COX II (including COX IIa and COX IIb) or COX III—two subunits of cytochrome c oxidase (e.g., from C. reinhardtii or Polytomella sp.); ATPase 6 from Chlorogonium elongatum and Chlamydomonas eugametos; and other peptides that are encoded by nuclear DNA in algae, and by mtDNA in mammals. Preferably, the peptide is Chlamydomonas reinhardtii ATPase 6 subunit of F₀F₁-ATP synthase.

The present invention further provides a method for treating a mitochondrial disorder in a subject in need of treatment for a mitochondrial disorder. The mitochondrial disorder may be any of those described above. In one embodiment of the present invention, the mitochondrial disorder is associated with a mutation (e.g., a point mutation) in mtDNA. Preferably, the mitochondrial disorder is FBSN, NARP, or MILS. A “subject”, as that term is used herein, is a mammal, and is preferably a human. The method of the present invention comprises administering to the subject a functional plant or protist (including algal) peptide in an amount effective to treat the mitochondrial disorder. The plant or protist peptide may be any peptide, including any of those disclosed herein. In one embodiment of the present invention, the peptide is a functional (e.g., wild-type) Chlamydomonas reinhardtii ATPase 6 subunit of F₀F₁-ATP synthase. In a preferred embodiment of the present invention, the mitochondrial disorder is FBSN, MILS, or NARP, and the peptide is a functional (e.g., wild-type) Chlamydomonas reinhardtii ATPase 6 subunit of F₀F₁-ATP synthase.

The phrase “effective to treat the mitochondrial disorder”, as used herein, means effective to ameliorate or minimize the clinical impairment or symptoms resulting from the mitochondrial disorder. For example, where the subject suffers from NARP, the clinical impairment or symptoms of the disorder may be ameliorated or minimized by diminishing or alleviating ataxia, discomfort, neuropathy, pain, or retinitis pigmentosa experienced by the subject. The amount of peptide effective to treat a mitochondrial disorder in a subject in need of treatment therefore will vary depending on the particular factors of each case, including the type of mitochondrial disorder, the stage of the mitochondrial disorder, the subject's age and weight, the severity of the subject's condition, and the method of administration. These amounts may be readily determined by the skilled artisan, using techniques known in the art and/or disclosed herein.

In accordance with the method of the present invention, the plant or protist (including algal) peptide may be administered to the subject by introducing into one or more cells of the subject a plant or protist (including algal) nucleic acid sequence (nuclear or non-nuclear) encoding the peptide, in a manner permitting expression of the peptide. Methods for carrying out this aspect of the present invention are described above. Without limitation, a plant or protist (including algal) nucleic acid sequence encoding the peptide of the present invention may be introduced into the cells of the subject (either in situ in the subject or ex vivo) by standard methods of transfection or transformation known in the art, including electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer, or any combination thereof. Recombinant viral vectors suitable for gene therapy include, but are not limited to, vectors derived from the genomes of viruses such as retrovirus, HSV, adenovirus, adeno-associated virus, Semiliki Forest virus, cytomegalovirus, and vaccinia virus.

Additionally, the plant or protist (including algal) peptide may be administered to the subject by a method comprising the steps of: (a) obtaining a nucleic acid sequence encoding the peptide; (b) preparing a nucleic-acid construct comprising a plant or protist (including algal) nucleic acid sequence encoding the peptide and, optionally, a plant or protist (including algal) nucleic acid sequence encoding a mitochondrial-targeting signal; (c) introducing the nucleic-acid construct into one or more cells of the subject; and (d) in at least one cell of the subject into which the nucleic-acid construct is introduced, expressing the nucleic-acid construct from the nucleus of the cell. The plant or protist (including algal) peptide that is expressed in the cytosol of the cell may then be targeted to, and introduced into, the mitochondrion under direction of the mitochondrial-targeting signal. In one embodiment of the present invention, step (b) is performed ex vivo.

Nucleic acid sequences for use in the method of the present invention may be isolated from cell cultures using known methods. Additional means for preparing the nucleic acid sequences have been described previously, and include, without limitation, restriction enzyme digestion of nucleic acid; and automated synthesis of oligonucleotides, using commercially-available oligonucleotide synthesizers such as the Applied Biosystems Model 392 DNA/RNA synthesizer. The mitochondrial-targeting signal—which has been previously described—may include a plant or protist (including algal) peptide sequence that occurs in nature, and which is added to a plant or protist (including algal) peptide that is generally transported to a mitochondrion. Additionally, the mitochondrial-targeting signal of the present invention may be an artificial or synthetic peptide sequence, which may correspond to a naturally-occurring transit sequence. Preferably, the mitochondrial-targeting signal is the mitochondrial-targeting signal of C. reinhardtii ATPase 6 subunit of F₀F₁-ATP synthase.

The present invention further provides an expression vector for use in introducing a functional peptide encoded by an algal nucleic acid sequence into a mitochondrion of a mammal. The phrase “expression vector” generally refers to nucleotide sequences that are capable of effecting expression of a structural gene in hosts compatible with such sequences. These expression vectors typically include at least suitable promoter sequences and, optionally, termination signals. The selection of suitable promoter sequences is well known in the art, as is the selection of appropriate expression vectors. See, e.g., Sambrook et al., Molecular Cloning: A Laboratory Manual (2d ed.), vols. 1-3 (Cold Spring Harbor, N.Y.: Cold Spring Harbor Laboratory, 1989). The expression vector of the present invention comprises a nucleic acid sequence encoding Chlamydomonas reinhardtii ATPase 6 subunit of F₀F₁-ATP synthase, or a nucleic acid sequence encoding the mitochondrial-targeting signal of Chlamydomonas reinhardtii ATPase 6 subunit of F₀F₁-ATP synthase (1-107aa of SEQ ID NO:1). As previously discussed in greater detail, the mitochondrial-targeting signal of the present invention may be derived from a peptide sequence that occurs in nature. Additionally, the mitochondrial-targeting signal of the present invention may be an artificial or synthetic peptide sequence, which may correspond to a naturally-occurring transit sequence.

As previously discussed, the expression vector of the present invention may be prepared by methods known in the art, including those described below. Promoters and ribosomal entry sites, including those disclosed herein, may be used in conjunction with standard techniques to prepare the expression vector of the present invention. Vectors that may be useful in the present invention include, without limitation, pCDNA3, bicistronic vectors (e.g., pEF-BOS-IRES), plasmid vectors (e.g., plasmid pcDNA3/5a-a vector), and adeno-associated virus (AAV) vectors (e.g., pTR-UF5, pTR-UF11, and pTR-UF12). Additionally, in accordance with the present invention, the expression vector may be labeled with a detectable marker, for facilitating detection of the Chlamydomonas reinhardtii ATPase 6 subunit of F₀F₁-ATP synthase. Labeling may be accomplished using one of a variety of labeling techniques, including any of those described herein. In a preferred embodiment of the present invention, the detectable marker is a FLAG epitope. This marker then may be detected using anti-FLAG antibodies and Western-blot analysis.

The present invention further provides a mammalian cell transformed by an expression vector for use in introducing a functional peptide encoded by a protist or plant (including algal) nucleic acid sequence into a mitochondrion, wherein the expression vector comprises a plant or protist (including algal) nucleic acid sequence encoding the peptide and, optionally, a plant or protist (including algal) nucleic acid sequence encoding a mitochondrial-targeting signal. As described above, the mammalian cell may be derived from any mammal, but is preferably a human cell. Moreover, the cell may be any type of cell, including, without limitation, a bone-marrow cell, a clonal cell, a germ-line cell, a post-mitotic cell (e.g., a cell of the central nervous system), a progenitor cell, and a stem cell. For example, the human cell line, 293T HEK, and the monkey COS7 kidney cell line, may be particularly useful in the practice of the present invention. In one embodiment of the present invention, the mammalian cell expresses a functional peptide. The present invention also provides clonal cell strains comprising the transformed mammalian cells described herein.

The present invention is also directed to a pharmaceutical composition, comprising: (a) a plant or protist (including algal) nucleic acid sequence encoding a peptide for introduction into a mitochondrion; (b) optionally, a plant or protist nucleic acid sequence encoding a mitochondrial-targeting signal; and (c) a pharmaceutically-acceptable carrier. Preferred plant or protist (including algal) nucleic acid sequences encoding the peptide for introduction into a mitochondrion, or encoding the mitochondrial-targeting signal, have been previously described.

The pharmaceutically-acceptable carrier must be “acceptable” in the sense of being compatible with the other ingredients of the composition, and not deleterious to the recipient thereof. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may be conveniently presented in unit dosage.

The formulations of the present invention may be prepared by methods well-known in the pharmaceutical art. For example, the nucleic acid sequences may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the nucleic acid sequences of the present invention to a subject to treat a mitochondrial disorder. The plant or protist (including algal) nucleic acid sequence encoding the peptide is provided in an amount that is effective to treat a mitochondrial disorder in the subject. That amount may be readily determined by the skilled artisan, as described above.

As discussed above, where the plant or protist (including algal) nucleic acid sequence encoding a peptide is non-nuclear (e.g., mtDNA), the peptide produced therefrom may not include a mitochondrial-targeting signal. However, where the plant or protist (including algal) nucleic acid sequence encoding a peptide is nuclear DNA, it may be expected that the peptide (e.g., precursor polypeptide) produced therefrom will include, or be contiguous with, a mitochondrial-targeting signal. Nevertheless, as shown herein, the unexpected ability of the mitochondrial-targeting signal (MTS) of C. reinhardtii ATPase 6 subunit of F₀F₁-ATP synthase to perform “xenotopic” expression or “trans-kingdom allotopic” expression may render this MTS particularly useful for importing highly hydrophobic proteins into human mitochondria (and, of equal importance, into mitochondria from model organisms, such as mice), even where such proteins already contain their own mitochondrial-targeting signals. Because the MTS of C. reinhardtii ATPase 6 subunit of F₀F₁-ATP synthase has been shown to be strong and efficient at targeting peptides to mitochondria, it is believed that it may be used to direct those peptides lacking an MTS, and, further, to supplement the MTSs of particularly hydrophobic proteins.

Accordingly, the present invention is also directed to a method for introducing a functional peptide into a mitochondrion, comprising the steps of: (a) preparing a nucleic-acid construct comprising a nucleic acid sequence encoding the peptide and a nucleic acid sequence encoding the mitochondrial-targeting sequence of Chlamydomonas reinhardtii ATPase 6 subunit of F₀F₁-ATP synthase; (b) introducing the nucleic-acid construct into a eukaryotic cell to produce a transformed cell; and (c) expressing the nucleic-acid construct from the nucleus of the transformed cell. The functional peptide that is expressed in the cytosol of the cell then may be targeted to, and introduced into, the mitochondrion under direction of the mitochondrial-targeting signal. The eukaryotic cell may be derived from an animal, a plant, a fungus, or a protozoan. In one embodiment of the present invention, the eukaryotic cell is a mammalian cell, including, without limitation, a bone-marrow cell, a germ-line cell, a post-mitotic cell (e.g., a cell of the central nervous system), a progenitor cell, and a stem cell. In a preferred embodiment, the cell is a human cell, including, without limitation, a cell from a human cell line (e.g., the human 293T HEK cell line).

In the method of the present invention, the peptide may be encoded by a nuclear or non-nuclear nucleic acid sequence, and may be any peptide from any organism. Preferably, the peptide is a mitochondrial peptide. In one embodiment of the present invention, the peptide is encoded by non-nuclear DNA (e.g., mtDNA). Examples of peptides encoded by mtDNA include, without limitation, apocytochrome b, an ATP synthase F₁ subunit, an ATP synthase F₀ subunit, a cytochrome c oxidase subunit, DNA polymerase, elongation factor, a haem lyase subunit, an NADH dehydrogenase subunit, an L ribosomal protein, an S ribosomal protein, RNA polymerase, an RNA polymerase subunit, reverse transcriptase, and succinate dehydrogenase subunit, as well as ATPase 8 subunit of F₀F₁-ATP synthase, an ATPase 6 subunit of F₀F₁-ATP synthase, and ATPase 6 of Prototheca wickerhamii, Pedinomonas minor, Cafeteria roenbergensis, and Chrysodidymus synuroideus. In a preferred embodiment, the peptide is human ATPase 6 subunit of F₀F₁-ATP synthase. In another embodiment of the present invention, the peptide is encoded by nuclear DNA. Examples of nuclear-DNA-encoded peptides for use in the present invention include, without limitation, an ATP synthase F₁ subunit, an ATP synthase F₀ subunit, a cytochrome c oxidase subunit, and an L ribosomal protein, as well as a subunit of NADH dehydrogenase ubiquinone oxidoreductase, the cytochrome b subunit of ubiquinone-cytochrome c oxidoreductase, and any of the nuclear-DNA-encoded peptides described above.

Where the peptide is encoded by a non-nuclear nucleic acid sequence, such as mtDNA, the method of the present invention may further comprise the step of modifying the non-nuclear nucleic acid sequence encoding the peptide, if necessary, before preparing the nucleic-acid construct (e.g., before step (a)), to render the non-nuclear nucleic acid sequence compatible with the universal genetic code, thereby permitting “allotopic expression” of the non-nuclear nucleic acid sequence. Techniques for modifying or mutagenizing nucleic acids are well-known in the art (Herlitze and Koenen, A general and rapid mutagenesis method using polymerase chain reaction. Gene, 91:143-47, 1990; Sutherland et al., Multisite oligonucleotide-mediated mutagenesis: application to the conversion of a mitochondrial gene to universal genetic code. Biotechniques, 18:458-64, 1995).

All organisms that have been studied to date, including both prokaryotes and eukaryotes, generally use the same code for synthesis of proteins by cytoplasmic ribosomes. The exception to this occurs in mitochondria. Like chloroplasts in plants, mitochondria contain their own genetic information, and are capable of carrying out both transcription and translation. The genetic system of mitochondria differs from other known genetic systems because it deviates from the standard, or “universal”, genetic code in several ways. In particular, the UGA codon, which generally means “stop”, codes for tryptophan in mammalian mitochondria; the AUA codon, which generally codes for isoleucine, codes for methionine in mammalian mitochondria; and the AGA codon, which generally codes for arginine, means “stop” in mammalian mitochondria. Accordingly, where a mitochondrial nucleic acid sequence is used in the method of the present invention, it may be necessary to first modify or mutagenize the nucleic acid sequence to render it compatible with the universal genetic code. In such instances, a modified or mutagenized mtDNA-specified polypeptide is appended to a mitochondrial-targeting signal, expressed from the nucleus, and transported back to the mitochondria under the guidance of the signal peptide.

The method of the present invention may be used to introduce a peptide into a mitochondrion in vitro, or in vivo in a mammal, by introducing the nucleic-acid construct of the present invention into a sufficient number of cells of the mammal (either in situ or ex vivo), in a manner permitting expression of the peptide encoded by the nucleic acid sequence contained within the construct. In view of the foregoing, the transformed eukaryotic cell of the present invention may be in, or introduced into, a mammal. The mammal may be any mammalian animal, but is preferably a human. Where the eukaryotic cell is already in a human, the mitochondrion may be contained within any cell of the human. Where ex vivo gene therapy techniques are used, the mitochondrion may be initially contained within a eukaryotic cell (including a bone-marrow, germ-line, post-mitotic, progenitor, or stem cell) outside of the human. The eukaryotic cell containing the functional peptide within the mitochondrion then may be introduced into the human (e.g., by standard techniques known in the art, including injection and transfusion) to permit in vivo proliferation of cells containing functional peptide within their mitochondria. Where ex vivo gene therapy techniques are used, the eukaryotic cell is preferably from a human, and, more preferably, from the same human as the one into whom the eukaryotic cell is introduced.

In accordance with the present invention, the use of allotopic expression to introduce a functional peptide encoded by a nucleic acid sequence into a mitochondrion may be utilized to rescue a mitochondrial disorder, such as a deficiency in ATP synthesis resulting from a defect in an mtDNA gene (e.g., MTATP6). Accordingly, the method of the present invention may be particularly useful for treating mitochondrial disorders. Thus, the present invention provides a method for treating a mitochondrial disorder in a human in need of treatment, comprising introducing to the human the nucleic-acid construct of the present invention. Examples of mitochondrial disorders have been described above. In one embodiment of the present invention, the mitochondrial disorder is associated with a mutation (e.g., a point mutation) in mtDNA. In another embodiment of the present invention, the mitochondrial disorder in the human is FBSN, MILS, or NARP, and the eukaryotic cell of the present invention (either in, or introduced into, the human) is transformed with a nucleic-acid construct comprising an mtDNA that encodes functional (e.g., wild-type) human ATPase 6 subunit of F₀F₁-ATP synthase.

The present invention is described in the following Examples, which are set forth to aid in the understanding of the invention, and should not be construed to limit in any way the scope of the invention as defined in the claims which follow thereafter.

### EXAMPLES

### Example 1

### Isolation of C. reinhardtii ATP6

Chlamydomonas reinhardtii strain 21 gr (mt+) (CC-1690) was obtained from the Chlamydomonas Genetic Center, Duke University, and was cultured under continuous light at room temperature (Snell, J. Cell Biol., 68:48-69, 1976). Using the Chlamydomonas EST Database (http://www.kazusa.orjp/en/plant/chlamy/EST; Asamizu et al., DNA Res., 6:369-73, 1999; Asamizu et al., DNA Res., 7:305-07, 2000), the inventors identified a number of overlapping ESTs encoding the putative ATP6 mRNA. Sets of oligonucleotides were designed on the basis of predicted overlapping ESTs, in order to amplify both the full-length cDNA and the chromosomal gene. Total RNA was extracted using standard methods (Wegener and Beck, Plant Mol. Biol., 16:937-46, 1991). First-strand cDNA was obtained using the SuperScript First Strand Synthesis System for RT-PCR (Gibco BRL, Gaithersburg, Md.). The SMART™ RACE cDNA Amplification Kit (Clontech, Palo Alto, Calif.) was used for determination of the 5′ and 3′ untranslated regions. Amplified PCR products were inserted into pCR®-TOPO vector (Invitrogen Corporation, Carlsbad, Calif.) for sequencing. Isolated clones were screened by automated sequencing using the ABI Prism Big Dye kit (Perkin Elmer, Foster City, Calif.). For amplification of the genomic sequence, genomic DNA was isolated by standard methods (Pan and Snell, J. Biol. Chem., 275:24106-114, 2000), and 1 μg of total DNA was used as a template in a PCR reaction using high-fidelity Taq polymerase (Roche Molecular Biochemicals, Indianapolis, Ind.). The C. reinhardtii ATP6 (CrATP6) cDNA and genomic sequences have been deposited in Genbank (accession numbers AF388174 and AY063772, respectively).

### Example 2

### Southern-Blot Hybridization

Ten μg of total genomic DNA was digested with appropriate restriction enzymes, separated through a 1% agarose gel, transferred onto nylon membranes (Schleicher & Schuell BioScience, Inc., Keene, N.H.), and probed with a random-primer-labeled PCR fragment—labeled with a Rapid Prime labeling kit (Roche Molecular Biochemicals)—corresponding to the CrATP6 coding region. Incubation of the probe with the membrane was carried out as previously described (Pan and Snell, J. Biol. Chem., 275:24106-114, 2000).

### Example 3

### Expression of CrATP6

Because no antibody to CrATP6 is known to be available, the inventors appended an in-frame sequence encoding a FLAG epitope tag (DYKDDDDK) (SEQ ID NO:5) to the 3′ end of the coding region of the full-length CrATP6 cDNA, and inserted the construct into the BamHI and EcoRI sites of the mammalian expression vector pCDNA3 (Invitrogen) using BamHI and EcoRI linkers flanking the insert. Positive clones were confirmed by sequencing. A positive plasmid (plasmid pcDNA3/5a-a, referred to herein as “pCrA6F” for clarity and brevity) was isolated using the Qiagen plasmid midi kit (Qiagen, Valencia, Calif.).

Transient transfections of human HEK 293T and monkey COS7 kidney cells were carried out with FuGENE 6 (Roche Molecular Biochemicals), a non-liposomal transfection reagent, according to the manufacturer's recommendations. Briefly, the DNA-FuGENE 6 complex was made fresh, at a ratio of 3 μl FuGENE to 1 μg plasmid DNA, in serum-free DMEM medium, overlain onto pre-plated cells, and incubated overnight at 37° C. Cells were cultured to confluency for further analyses. For stable transfections of human cybrids JCP213 (100% wild-type mtDNA; 8993T) and JCP261 (100% mutant mtDNA; 8993G), cells were transfected as described above; upon confluency, the neomycin analog, G418, was added to the high-glucose DMEM growth medium (Gibco—Life Technologies, Inc., Gaithersburg, Md.) to select for positive cells. Stable transfections were maintained in medium with G418 for 4 weeks before growth experiments were carried out.

For growth in glucose, cells were grown in DMEM medium supplemented with 5% fetal bovine serum, 2 mM L-glutamine, and 4.5 mg/ml glucose. For growth in galactose, the above medium was used, except that 5 mM galactose was substituted for glucose; the cells were left in this medium for 4 days before being changed to the glucose medium. To determine the sensitivity to oligomycin (Manfredi et al., J. Biol. Chem., 274:9386-91, 1991), cells were grown in galactose medium containing 0.1 ng/ml oligomycin for 4 days; the medium was then changed to glucose for recovery.

ATP synthesis was measured as described (James et al., Eur. J. Biochem., 259:462-69, 1999). Briefly, cells were incubated in 150 mM KCl, 25 mM TrisHCl (pH 7.4), 2 mM EDTA, 10 mM potassium phosphate, 0.1 mM MgCl₂, and 0.1 % BSA, with 50 μg/ml digitonin. Mitochondria were energized using 1 mM malate and 1 mM pyruvate as substrates, in the presence of 1 mM ADP and 0.15 mM of the adenylate kinase inhibitor, P¹,P⁵-di(adenosine)pentaphosphate, and incubated for 10 min at 37° C. A 50-μl aliquot was resuspended in 25 mM TrisHCl (pH 7.4), and boiled for 2 min. Parallel incubations were also carried out in the presence of 2 μg/ml oligomycin to measure mitochondrial-specific ATP synthesis. The supernatant was assayed for ATP using a luciferin-luciferase assay (Manfredi et al., Methods Cell Biol., 65:133-45, 2001) with an ATP bioluminescence assay kit CLSII (Roche Molecular Biochemicals) in an Optocomp-1 luminometer (MGM Instruments, Hamden, Conn.).

### Example 4

### Immunological Techniques

Co-localization of the expressed protein with mitochondria was assessed by treatment of the transfected cells (grown on coverslips) with 500 nM of the mitochondrial-specific fluorescent dye, Mitotracker® Red (Molecular Probes, Inc., Eugene, Oreg.), and by immunodetection of the FLAG epitope with an anti-FLAG M2 primary IgG antibody and a goat anti-mouse IgG secondary antibody conjugated to Oregon green (Molecular Probes, Inc.). Briefly, the cells were incubated with Mitotracker, diluted in basic DMEM medium at 37° C., washed in PBS, fixed with 4% paraformaldehyde (w/v), and permeabilized with cold acetone. Nonspecific conjugation was blocked with non-immune goat serum in PBS. Coverslips were mounted on slides with an aqueous mounting medium (Biomeda Corp., Foster City, Calif.). Immunofluorescence was visualized using an Olympus IX70 microscope, and images were captured using an RT color SPOT digital camera (Diagnostic Instruments, Sterling Heights, Mich.).

Expression from transfected pCRA6F was detected by Western blotting. Briefly, cells were harvested, washed in phosphate-buffered saline (1× PBS; Gibco—Life Technologies, Inc.), and homogenized in mitochondrial isolation buffer (220 mM mannitol; 70 mM sucrose; 1 mM EDTA, pH 7.4). Crude mitochondria were isolated as described previously (Ojaimi et al., Mech. Ageing Dev., 111:39-47, 1999). Protein samples at a concentration of 10 μg of total homogenate, as well as isolated mitochondria, were electrophoresed through a 15% SDS-PAGE gel, and electro-transferred onto a polyvinyldine difluoride (PVDF) membrane (Bio-Rad Laboratories, Hercules, Calif.). For blue-native PAGE, 20 μg crude mitochondria were solubilized by sonication, and electophoresed through a 5-18% continuous gradient gel under non-denaturing conditions. Lanes from the first dimension (blue native) were cut out, treated to denature the protein subunits, and inserted horizontally into a denaturing 15% SDS-PAGE gel for analysis in the second dimension. Proteins from the first and second dimensions were electro-transferred onto a PVDF membrane. Expressed proteins were detected using a mouse monoclonal antibody against the FLAG epitope, anti-Flag M2 (Sigma Immunochemicals, St. Louis, Mo.), and were visualized using the ECL-Plus chemiluminescence system (Amersham Pharmacia, Piscataway, N.J.).

Isolation and Characterization of Chlamydomonas reinhardtii ATP6

In the course of their studies, the inventors initially designed degenerate primers corresponding to the region of ATP6 that is most conserved among various species, in order to amplify subregions of the putative CrATP6 gene from both C. reinhardtii mRNA and C. reinhardtii genomic DNA. Unfortunately, however, these attempts were unsuccessful.

Using the translation product from the mtDNA-encoded ATP6 gene from another algal species, Prototheca wickerhamii (Genbank accession number U02970), to screen the Chlamydomonas EST database, the inventors identified three overlapping ESTs (Genbank accession numbers BE121716, AV623443, and AV621415) corresponding to the putative full-length CrATP6 mRNA. Using these cDNAs as templates, the inventors eventually assembled a 1079-bp CrATP6 cDNA containing 31 nucleotides (nt) of the 5′ untranslated region (5′ UTR), a 1020-nt open reading frame specifying a 340-amino-acid (aa) polypeptide, and at least 28 nt of the 3′ untranslated region (3′ UTR) of the ATP6 mRNA (FIG. 1A). Using PCR primers from regions of this cDNA to amplify C. reinhardtii total genomic DNA, the inventors obtained a 2222-bp fragment representing the ATP6 gene (FIG. 1A).

During the course of this work, Funes et al. (J. Biol. Chem., 277:6051-58, 2002) also reported the isolation of a full-length CrATP6 cDNA (Genbank accession number AF411119) and gene (Genbank accession number AF411921). That cDNA, which is 2,349 bp in length, is essentially identical to that reported here, but has an additional 1.3 kb of 3′ UTR sequence. CrATP6 is almost certainly a single-copy gene (Funes et al., supra), as Southern-blot hybridization analysis has revealed that it is contained on a single EcoRI and SphI fragment, and, as predicted by the gene sequence, on only two MboI fragments (FIG. 1B).

The deduced translation product of CrATP6 (FIG. 1C) is 340 amino acids long (with a predicted molecular mass of 35.5 kDa), and the first 107 amino acids constitute the MTS (Funes et al., J. Biol. Chem., 277:6051-58, 2002). In the 233-aa mature polypeptide (predicted molecular mass of 24.6 kDa), the N-terminal 60 amino acids have a very low degree of primary-sequence identity with the mtDNA-encoded ATPase 6 polypeptides from highly diverse organisms. The remaining C-terminal residues are more conserved (FIG. 1C), not only in the related algae Prototheca wickerhamii (about 42% identity) and Pedinomonas minor (33%), but also in Saccharomyces cerevisiae (37%) and Homo sapiens (32%), and even in Escherichia coli (25%) and ATPI—the analogous subunit of the ATP synthase in C. reinhardtii chloroplasts (22%). In spite of the low degree of sequence identity overall, the conservation of secondary structure along the entire length of these orthologous polypeptides is remarkably high (FIG. 1A), presumably reflecting the evolutionary constraints on the requirement of this subunit to be able to couple proton flow to the rotation of the ring of c subunits with which it makes contact (Rastogi and Girvin, Nature, 402:263-68, 1999).

Expression of C. reinhardtii ATPase 6 in Mammalian Cells

The high conservation of secondary structure described above encouraged the inventors to determine whether CrATP6 could be expressed and targeted to mitochondria in mammalian cells. Since no antibodies to CrATP6 are available, the inventors appended sequences encoding a FLAG epitope tag to the C terminus of the full-length CrATP6 cDNA (referred to as “CrATP6F”), and inserted this construct into pCDNA3, a mammalian expression vector (referred to herein as plasmid “pCrA6F”). Transient expression of pCrA6F in both SV40-transformed monkey COS7 cells and human 293T HEK kidney cells showed that the expressed CrATP6F protein was targeted to mitochondria (FIGS. 2A and 2B). Western-blot analysis of both total cell homogenate and purified mitochondria isolated from 293T cells confirmed that CrATP6F was targeted to mitochondria (FIG. 3A). Moreover, the precursor protein (predicted size 36.5 kDa) was imported into the mitochondria, and was processed to produce the presumably mature polypeptide, with a size of 26 kDa. (The predicted size of mature CrATP6F is 25.6 kDa.)

As it is difficult to deduce the molecular mass of highly hydrophobic proteins, such as CrATP6F, in SDS-PAGE gels (Mariottini et al., Proc. Natl. Acad. Sci. USA, 83:1563-67, 1986), the inventors cannot know with any degree of certainty whether the site of cleavage of the CrATP6F precursor in human mitochondria is the same as that of CrATP6 in algal mitochondria. However, the approximately 10-kDa difference in the sizes of the precursor and the mature polypeptides (FIG. 3A) implies that the cleavage site is, in fact, quite close to the expected position at aa-107, suggesting that the unusually long and presumably complex MTS (Daley et al., Plant J., 30:11-21, 2002) of CrATP6F is “recognized” by the human importation machinery.

The amount of mature polypeptide that was expressed in the cells was approximately 40% of the total expressed protein, implying that importation of CrATP6F into human mitochondria was relatively inefficient. This is similar to what the inventors observed upon allotopic expression of human ATPase 6 (Manfredi et al., Nature Genet., 25:394-99, 2002). The inventors believe that the unprocessed precursors that were present at relatively high levels in the isolated mitochondrial fraction were either loosely attached to the mitochondrial outer membrane, or attached but not efficiently imported.

Analysis using a combination of blue-native and SDS gels (Schägger et al., Anal. Biochem., 173:201-05, 1988; Schagger and Ohm, Eur. J. Biochem., 227:916-21, 1995) strongly implied that the imported and processed CrATP6F was incorporated into human F₀F₁-ATP synthase (FIG. 3B). In particular, use of anti-FLAG antibodies to probe a Western blot of proteins from 293T cells transfected transiently with pCrA6F, and separated on a non-denaturing blue-native gel, revealed a signal migrating at approximately 600 kDa, consistent in size with that of complex V, as previously demonstrated by the inventors (Manfredi et al., Nature Genet., 25:394-99, 2002) (FIG. 3B, left panel). Furthermore, when the lane containing this band was run in the second dimension on a denaturing SDS gel, the anti-FLAG signal was then detected at the appropriate lateral position on the gel, and with an estimated molecular mass of 26 kDa (FIG. 3B, right panel). This result implies that the mature 26-kDa CrATP6F polypeptide was indeed incorporated into human complex V.

Two other minor spots, also in the size range of 26-27 kDa, were observed in the second dimension. The horizontal distance separating the main spot from these two minor spots implied that some anti-FLAG-reactive material had been present in the blue-native gel in a complex of about 500 kDa. The inventors do not know the origin of these two spots, as their sizes are not consistent with those estimated for either the F₀ (˜200 kDa) or F₁ (˜400 kDa) subcomplexes of ATP synthase.

Expression of CrATP6F in Human Cybrids Harboring the T8993G Mutation

Having shown that ATPase 6 from Chlamydomonas reinhardtii could assemble into human complex V, the inventors wanted to determine whether such a “chimeric” ATP synthase could function in human mitochondria. As noted above, the inventors previously showed that allotopic expression of a genetically-modified, nucleus-localized version of the human ATP6 gene (which is normally mtDNA-encoded) was capable of rescuing a deficiency in ATP synthesis in cytoplasmic hybrid (cybrid) cells harboring homoplasmic levels of the T8993G mutation found in NARP and MILS patients (Manfredi et al., Nature Genet., 25:394-99, 2002). Therefore, the inventors carried out a similar analysis using the Chlamydomonas construct. To do this, the inventors transfected wild-type (i.e., 100% 8993T) and mutant (i.e., 100% 8993G) cybrids with pCrA6F, and used selection for resistance to the neomycin analog, G418, to isolate stably-transfected cells.

The inventors confirmed that the expressed CrATP6F was targeted to the mitochondria of stably-transfected wild-type (not shown) and mutant cybrids (FIG. 2C), as was the case with the transiently-transfected cells. After one month in culture, the inventors monitored the growth rate of the cells. The inventors determined that the mutant cybrids grew as well in rich medium containing glucose as did the wild-type cybrids (FIG. 4, upper panel), and were only slightly affected when grown in medium containing galactose instead of glucose (FIG. 4, middle panel). This is similar to what the inventors previously demonstrated (Manfredi et al., J. Biol. Chem., 274:9386-91, 1991). Previously, the inventors also showed that, in a growth medium containing galactose plus low levels of oligomycin, which binds specifically to ATPase 6 (Breen et al., J. Biol. Chem., 261:11680-685, 1986; John and Nagley, FEBS Lett., 207:79-83, 1986) and inhibits complex V function, the growth of cells containing mutant mtDNA is inhibited compared to that of wild-type cells (Manfredi et al., J. Biol. Chem., 274:9386-91, 1991) or that of mutant cybrids in which human ATPase 6 was expressed allotopically (Manfredi et al., Nature Genet., 25:394-99, 2002).

The inventors observed that, in the presence of galactose plus oligomycin, the stably-transfected mutant lines that express CrATP6F grew better than did the untransfected mutant cybrids, although growth of the stably-transfected mutant lines was somewhat less than that of the wild-type cybrids (FIG. 4, lower panel). The inventors measured mitochondrial (i.e., oligomycin-sensitive) ATP synthesis in triplicate platings of cells from two independent stable transfections of pCrA6F in wild-type and mutated cybrids, as well as from untransfected control cybrids grown in parallel (FIG. 5). The level of ATP synthesis in mutant cybrids prior to transfection was approximately 35% of the level in wild-type cybrids—a value that is consistent with that which was observed previously in patient cells (Manfredi et al., J. Biol. Chem., 274:9386-91, 1991; Tatuch and Robinson, Biochem. Biophys. Res. Commun., 192:124-28, 1993; Vazquez-Memije et al., J. Inher. Metab. Dis., 19:43-50, 1996; Garcia et al., J. Biol. Chem., 275:11075-081, 2000; Schon et al., Semin. Cell Dev. Biol., 12:441-48, 2001; Manfredi et al., Nature Genet., 25:394-99, 2002). In the two wild-type cybrids transfected with pCrA6F, ATP synthesis remained unchanged in one transfection; yet, for unknown reasons, ATP synthesis increased in the second transfection, as compared to the control wild-type cybrids. Interestingly, the inventors did not detect any diminution in ATP synthesis in these cells, implying that the expressed CrATP6F had little, if any, negative effects on the functioning of complex V.

In the two mutant cybrids transfected with pCrA6F, mitochondrial ATP synthesis increased above control values by approximately 75%. However, the amount of ATP synthesis in the transfected mutant cybrids was still less than that in the wild-type cybrids (FIG. 5). In other words, while the expression of CrATP6F in mutant cybrids almost doubled the ability of those cells to generate ATP, this improvement was still well below normal levels. It should be noted, though, that expression of the C. reinhardtii ATP6F polypeptide resulted in an increase in ATP synthesis in the mutant cells, despite the fact that endogenously-synthesized mutant human mtDNA-encoded ATPase 6 polypeptides were competing with the algal polypeptides for assembly into complex V holoproteins. Importantly, this degree of improvement in oxidative energy metabolism was clearly sufficient to allow the cells to grow in the galactose-oligomycin medium (FIG. 4).

Evolutionary Considerations and Implications for the Future

The endosymbiont hypothesis for the origin of mitochondria (Lang et al., Annu. Rev. Genet., 33:351-97, 1999) implies that more than 99% of the approximately 4,000 genes originally present in the proto-mitochondrion are not present in the organelle today. Many of these genes were presumably non-essential to the viability of the eukaryotic host, and were lost during the course of evolution; the remainder were transferred to the nucleus, and have become the ancestors of many of the “housekeeping” genes present in modern eukaryotes (Lang et al., supra; Gray et al., Genome Biol., 2:1018.1011-1015, 2001). Most of the ancestor proteins have evolved to be retargeted back to mitochondria, where they comprise the majority of the estimated 850 gene products present in the modern mammalian organelle.

The evolution of organellar genetic codes differing from the universal code cemented the division between nuclear-DNA-encoded and mitochondrial-DNA-encoded polypeptides. However, a tiny subset of proteins, all components of the mitochondrial respiratory chain/oxidative phosphorylation system, escaped this massive gene transfer process, and remained in the mitochondrial genome. With but a few exceptions, a “canonical” set of mtDNA-encoded proteins—typically six subunits of NADH dehydrogenase ubiquinone oxidoreductase, the cytochrome b subunit of ubiquinone-cytochrome c oxidoreductase, three subunits of cytochrome c oxidase, and two subunits of ATP synthase—is remarkably conserved among all examined species, ranging from the protist Reclinomonus americana to mammals. While the reason for this conservation has been the subject of speculation, the most widely-held view is that these proteins are so hydrophobic that they are unable to be imported from the cytoplasm; thus, this set was constrained by evolutionary pressure to remain in the mitochondrial genome (Claros et al., Eur. J. Biochem., 228:762-71, 1995; Claros et al., Meth. Enzymol., 264:389-403, 1996; Perez-Martinez et al., J. Biol. Chem., 275:30144-152, 2000; Perez-Martinez et al., J. Biol. Chem., 276:11302-309, 2001).

Despite the foregoing, the “rules” determining which hydrophobic-protein-encoding genes were retained in the mtDNA, and which were transferred to the nucleus, are not hard and fast. For example, the mtDNAs of the yeasts Saccharomyces cerevisiae and Schizosaccharomyces pombe contain the ATP9 gene, which encodes subunit c of ATP synthase; in contrast, subunit c of ATP synthase is nucleus-encoded in mammals. Even more striking has been the finding that some organisms among the algae, ciliates, apicomplexans, and flowering plants lack mtDNA-encoded COX II, COX III, and/or ATP6—all highly hydrophobic proteins. Since these three subunits are necessary for the functioning of COX and ATP synthase, respectively, it is almost certain that these genes have been transferred to the nuclear DNA in these organisms. In fact, the nucleus-encoded genes specifying COX II (including COX IIa and COX IIb), COX III (Perez-Martinez et al., J. Biol. Chem., 275:30144-152, 2000; Perez-Martinez et al., J. Biol. Chem., 276:11302-309, 2001; Watanabe and Ohama, J. Mol. Evol., 53:333-39, 2001), and, as reported here and elsewhere (Funes et al., J. Biol. Chem., 277:6051-58, 2002), ATPase 6, have been identified in algal species, including C. reinhardtii and Polytomella sp.

It is noteworthy that the mitochondrial genetic code of C. reinhardtii is identical to the universal nuclear code (Boer and Gray, Curr. Genet., 14:583-90, 1988). Presumably, over the course of evolution, the presence of a nuclear-DNA-compatible genetic code allowed for a more facile transfer of mtDNA-encoded genes to the nucleus, but this does not mean that all such mtDNA-encoded genes can be transferred. As discussed in some detail by the Gonzalez-Halphen group (Perez-Martinez et al., J. Biol. Chem., 275:30144-152, 2000; Perez-Martinez et al., J. Biol. Chem., 276:11302-309, 2001; Funes et al., J. Biol. Chem., 277:6051-58, 2002), COX II, COX III, and ATPase 6 all reside at the lower end of the scale of “meso-hydrophobicity” of mtDNA-encoded polypeptides, whereas COX I and cytochrome b—which have never been found to be nucleus-encoded in any examined organism—reside at the high end. Thus, the relatively lower degree of hydrophobicity of ATPase 6, coupled with a compatible genetic code, fortuitously allowed for CrATP6's transfer to, and ultimate re-targeting back to, the mitochondria.

Although mammals and algae are separated by more than a billion years of evolution, the inventors were able to demonstrate that the nucleus-encoded Chlamydomonas reinhardtii ATPase 6 polypeptide can function in human mitochondria. In particular, the algal CrATP6 gene, when expressed in human cybrids harboring a homoplasmic pathogenic mutation in the analogous mtDNA-encoded MTATP6 gene, enabled these cells to be viable under growth conditions (i.e., galactose plus oligomycin) that killed the untransfected mutant cells. This rescue of viability was almost certainly due to an increase in the ability of the cybrids to produce enough ATP to be maintained in tissue culture. The inventors note, however, that the complementation of function by CrATP6F was only moderate, as the transfected cells still grew at a rate almost ten-fold lower than that of wild-type cybrids. It is likely that much of this reduced rate resulted from the presence within the homoplasmic mutant cybrid mitochondria of complex V holoproteins containing the “endogenous” mutated human ATPase 6 subunit. Given the competition between the human and C. reinhardtii ATPase 6 subunits for assembly into complex V holoproteins, the inventors deem it rather remarkable that the algal ATPase 6 could replace its human homolog as part of a chimeric F₀F₁-ATP synthase. The recessive nature of the NARP/MILS T8993G mutation implies that even a partial rescue of function will be of clinical utility.

The inventors recently showed that allotopic expression of the human MTATP6 gene could rescue deficiencies in cell growth and in ATP synthesis in cybrids harboring homoplasmic levels of the 8993G mutation (Manfredi et al., Nature Genet., 25:394-99, 2002). The work reported here extends the potential application of successful allotopic expression of “cognate” gene products (e.g., an allotopically-expressed, recoded human mitochondrial gene targeted to human mitochondria) to the expression of homologous gene products across species boundaries, similar to the rescue of deficiencies in human (Bai et al., J. Biol. Chem., 276:38808-813, 2001) and hamster (Seo et al., Proc. Natl. Acad. Sci. USA, 95:9167-71, 1998) rotenone-sensitive complex I by the expression of the nucleus-encoded, rotenone-insensitive NADH quinone oxidoreductase gene (NDI1) from Saccharomyces cerevisiae. The ability to perform this type of “xenotopic” expression or “trans-kingdom” allotopic expression may be particularly useful in efforts to import highly hydrophobic proteins into human mitochondria (and, of equal importance, into mitochondria from model organisms, such as mice). For example, the MTS of CrATP6 might be capable of directing the importation into mitochondria of a human cytochrome b subunit (which is normally mtDNA-encoded) that has been engineered specifically for allotopic expression in cells from patients harboring mutations in this polypeptide (Rana et al., Ann. Neurol., 48:774-81, 2000).

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for introducing a functional peptide encoded by a plant or protist nucleic acid sequence into a mitochondrion of a mammalian cell, comprising the steps of:
(a) preparing a nucleic-acid construct comprising a plant or protist nucleic acid sequence encoding the peptide and, optionally, a plant or protist nucleic acid sequence encoding a mitochondrial-targeting signal;
(b) introducing the nucleic-acid construct into a mammalian cell to produce a transformed cell; and
(c) expressing the nucleic-acid construct from the nucleus of the transformed cell.

2. The method of claim 1, wherein the peptide is a nuclear-DNA-encoded peptide.

3. The method of claim 1, wherein the plant or protist nucleic acid sequence encoding the peptide is an algal nucleic acid sequence.

4. The method of claim 3, wherein the peptide is Chlamydomonas reinhardtii ATPase 6 subunit of F₀F₁-ATP synthase.

5. The method of claim 1, wherein the mitochondrial-targeting signal (MTS) is the MTS of Chlamydomonas reinhardtii ATPase 6 subunit of F₀F₁-ATP synthase.

6. The method of claim 1, wherein the mammalian cell is a human cell.

7. The method of claim 6, where the cell is a human 293T HEK cell.

8. The method of claim 1, wherein the nucleic-acid construct is introduced into the mammalian cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

9. The method of claim 1, wherein the nucleic-acid construct further comprises a nucleic acid sequence encoding a detectable marker.

10. The method of claim 9, wherein the detectable marker is a FLAG epitope.

11. The method of claim 1, wherein the peptide is Chlamydomonas reinhardtii ATPase 6 subunit of F₀F₁-ATP synthase and the mammalian cell is a human cell.

12. The method of claim 1, wherein the mammalian cell is in, or is introduced into, a human.

13. The method of claim 12, wherein the human has a mitochondrial disorder.

14. The method of claim 13, wherein the mitochondrial disorder is associated with a mutation in mtDNA.

15. The method of claim 14, wherein the mutation is a point mutation.

16. The method of claim 14, wherein the mitochondrial disorder is selected from the group consisting of FBSN (familial bilateral striatal necrosis), NARP (neuropathy, ataxia, and retinitis pigmentosa), and MILS (maternally-inherited Leigh syndrome).

17. The method of claim 16, wherein the peptide is ATPase 6 subunit of F₀F₁-ATP synthase.

18. A method for correcting a phenotypic deficiency in a mammal that results from a mutation in a mitochondrial peptide, comprising the steps of:
(a) establishing the identity of the mitochondrial peptide having the mutation;
(b) preparing a nucleic-acid construct comprising a plant or protist nucleic acid sequence encoding the peptide and, optionally, a plant or protist nucleic acid sequence encoding a mitochondrial-targeting signal, wherein the plant or protist nucleic acid sequence encoding the peptide encodes a functional peptide;
(c) introducing the nucleic-acid construct into a mammalian cell to produce a transformed cell; and
(d) expressing the nucleic-acid construct from the nucleus of the transformed cell.

19. The method of claim 18, wherein the peptide is a nuclear-DNA-encoded peptide.

20. The method of claim 18, wherein the plant or protist nucleic acid sequence encoding the peptide is an algal nucleic acid sequence.

21. The method of claim 20, wherein the peptide is Chlamydomonas reinhardtii ATPase 6 subunit of F₀F₁-ATP synthase.

22. The method of claim 18, wherein the mitochondrial-targeting signal (MTS) is the MTS of Chlamydomonas reinhardtii ATPase 6 subunit of F₀F₁-ATP synthase.

23. The method of claim 18, wherein the mammalian cell is a human cell.

24. The method of claim 18, wherein the nucleic-acid construct is introduced into the mammalian cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

25. The method of claim 18, wherein the peptide is Chlamydomonas reinhardtii ATPase 6 subunit of F₀F₁-ATP synthase and the mammalian cell is a human cell.

26. The method of claim 18, wherein the mammalian cell is in, or is introduced into, a human.

27. The method of claim 26, wherein the human has a mitochondrial disorder.

28. The method of claim 27, wherein the mitochondrial disorder is associated with a mutation in mtDNA.

29. The method of claim 28, wherein the mutation is a point mutation.

30. The method of claim 28, wherein the mitochondrial disorder is selected from the group consisting of FBSN (familial bilateral striatal necrosis), NARP (neuropathy, ataxia, and retinitis pigmentosa), and MILS (maternally-inherited Leigh syndrome).

31. The method of claim 30, wherein the peptide is ATPase 6 subunit of F₀F₁-ATP synthase.

32. A method for treating a mitochondrial disorder in a subject in need of treatment therefore, comprising administering to the subject a functional plant or protist peptide in an amount effective to treat the mitochondrial disorder.

33. The method of claim 32, wherein the subject is a mammal.

34. The method of claim 33, wherein the mammal is a human.

35. The method of claim 32, wherein the peptide is a nuclear-DNA-encoded peptide.

36. The method of claim 32, wherein the plant or protist is an alga.

37. The method of claim 36, wherein the peptide is Chlamydomonas reinhardtii ATPase 6 subunit of F₀F₁-ATP synthase.

38. The method of claim 32, wherein the mitochondrial disorder is associated with a mutation in mtDNA.

39. The method of claim 38, wherein the mutation is a point mutation.

40. The method of claim 38, wherein the mitochondrial disorder is selected from the group consisting of FBSN (familial bilateral striatal necrosis), NARP (neuropathy, ataxia, and retinitis pigmentosa), and MILS (maternally-inherited Leigh syndrome).

41. The method of claim 40, wherein the peptide is Chlamydomonas reinhardtii ATPase 6 subunit of F₀F₁-ATP synthase.

42. The method of claim 32, wherein the peptide is administered to the subject by introducing into one or more cells of the subject a nucleic acid sequence encoding the peptide, in a manner permitting expression of the peptide.

43. The method of claim 32, wherein the peptide is administered to the subject by a method comprising the steps of:
(a) obtaining a nucleic acid sequence encoding the peptide;
(b) preparing a nucleic-acid construct comprising a plant or protist nucleic acid sequence encoding the peptide and, optionally, a nucleic acid sequence encoding a mitochondrial-targeting signal;
(c) introducing the nucleic-acid construct into one or more cells of the subject; and
(d) in at least one cell of the subject into which the nucleic-acid construct is introduced, expressing the nucleic-acid construct from the nucleus of the cell.

44. The method of claim 43, wherein step (c) is performed ex vivo.

45. The method of claim 43, wherein the peptide is Chlamydomonas reinhardtii ATPase 6 subunit of F₀F₁-ATP synthase.

46. The method of claim 43, wherein the mitochondrial-targeting signal (MTS) is the MTS of Chlamydomonas reinhardtii ATPase 6 subunit of F₀F₁-ATP synthase.

47. The method of claim 43, wherein the nucleic-acid construct is introduced into one or more cells of the subject by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

48. An expression vector for use in introducing a functional peptide encoded by an algal nucleic acid sequence into a mitochondrion of a mammal, comprising a nucleic acid sequence encoding Chlamydomonas reinhardtii ATPase 6 subunit of F₀F₁-ATP synthase or the mitochondrial-targeting signal thereof.

49. The expression vector of claim 48, further comprising a nucleic acid sequence encoding a detectable marker.

50. The expression vector of claim 49, wherein the detectable marker is a FLAG epitope.

51. The expression vector of claim 48, wherein the vector is selected from the group consisting of a bicistronic vector, a plasmid vector, and an adeno-associated virus (AAV) vector.

52. A mammalian cell transformed by the expression vector of claim 48.

53. A mammalian cell transformed by the expression vector of claim 50.

54. A mammalian cell transformed by an expression vector for use in introducing a functional peptide encoded by a plant or protist nucleic acid sequence into a mitochondrion, wherein the expression vector comprises a plant or protist nucleic acid sequence encoding the peptide and, optionally, a plant or protist nucleic acid sequence encoding a mitochondrial-targeting signal.

55. The mammalian cell of claim 54, wherein the cell expresses the peptide.

56. The mammalian cell of claim 54, which is a human cell.

57. The mammalian cell of claim 54, which is selected from the group consisting of a clonal cell, a stem cell, and a progenitor cell.

58. The mammalian cell of claim 54, wherein the peptide is a nuclear-DNA-encoded peptide.

59. The mammalian cell of claim 54, wherein the plant or protist nucleic acid sequence encoding the peptide is an algal nucleic acid sequence.

60. The mammalian cell of claim 59, wherein the peptide is Chlamydomonas reinhardtii ATPase 6 subunit of F₀F₁-ATP synthase.

61. The mammalian cell of claim 54, wherein the mitochondrial-targeting signal (MTS) is the MTS of Chlamydomonas reinhardtii ATPase 6 subunit of F₀F₁-ATP synthase.

62. The mammalian cell of claim 54, wherein the expression vector transforms the cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

63. The mammalian cell of claim 54, wherein the expression vector further comprises a nucleic acid sequence encoding a detectable marker.

64. The mammalian cell of claim 63, wherein the detectable marker is a FLAG epitope.

65. The mammalian cell of claim 54, wherein the expression vector is selected from the group consisting of a bicistronic vector, a plasmid vector, and an adeno-associated virus (AAV) vector.

66. A clonal cell strain comprising the transformed mammalian cell of claim 54.

67. A pharmaceutical composition, comprising:
(a) a plant or protist nucleic acid sequence encoding a peptide for introduction into a mitochondrion;
(b) optionally, a plant or protist nucleic acid sequence encoding a mitochondrial-targeting signal; and
(c) a pharmaceutically-acceptable carrier.

68. The pharmaceutical composition of claim 67, wherein the peptide is a nuclear-DNA-encoded peptide.

69. The pharmaceutical composition of claim 68, wherein the plant or protist nucleic acid sequence encoding a peptide for introduction into a mitochondrion is an algal nucleic acid sequence.

70. The pharmaceutical composition of claim 69, wherein the peptide is Chlamydomonas reinhardtii ATPase 6 subunit of F₀F₁-ATP synthase.

71. The pharmaceutical composition of claim 67, wherein the mitochondrial-targeting signal (MTS) is the MTS of Chlamydomonas reinhardtii ATPase 6 subunit of F₀F₁-ATP synthase.

72. A method for introducing a functional peptide into a mitochondrion, comprising the steps of:
(a) preparing a nucleic-acid construct comprising a nucleic acid sequence encoding the peptide and a nucleic acid sequence encoding the mitochondrial-targeting sequence of Chlamydomonas reinhardtii ATPase 6 subunit of F₀F₁-ATP synthase;
(b) introducing the nucleic-acid construct into a eukaryotic cell to produce a transformed cell, wherein the eukaryotic cell is derived from an animal, a plant, a fungus, or a protozoan; and
(c) expressing the nucleic-acid construct from the nucleus of the transformed cell.

73. The method of claim 72, wherein the peptide is encoded by mitochondrial DNA.

74. The method of claim 73, further comprising the step of modifying the mitochondrial DNA (mtDNA), if necessary, before step (a), to render the mtDNA compatible with the universal genetic code.

75. The method of claim 74, wherein the peptide is selected from the group consisting of apocytochrome b, an ATP synthase F₁ subunit, an ATP synthase F₀ subunit, a cytochrome c oxidase subunit, DNA polymerase, elongation factor, a haem lyase subunit, a NADH dehydrogenase subunit, an L ribosomal protein, an S ribosomal protein, RNA polymerase, an RNA polymerase subunit, reverse transcriptase, and succinate dehydrogenase subunit.

76. The method of claim 75, wherein the peptide is human ATPase 6 subunit of F₀F₁-ATP synthase.

77. The method of claim 72, wherein the peptide is a nuclear-DNA-encoded peptide.

78. The method of claim 77, wherein the peptide is selected from the group consisting of an ATP synthase F₁ subunit, an ATP synthase F₀ subunit, a cytochrome c oxidase subunit, and an L ribosomal protein.

79. The method of claim 72, wherein the nucleic-acid construct is introduced into the eukaryotic cell by a method selected from the group consisting of electroporation, DEAE Dextran transfection, calcium phosphate transfection, cationic liposome fusion, protoplast fusion, creation of an in vivo electrical field, DNA-coated microprojectile bombardment, injection with a recombinant replication-defective virus, homologous recombination, ex vivo gene therapy, a viral vector, and naked DNA transfer.

80. The method of claim 72, wherein the nucleic-acid construct further comprises a nucleic acid sequence encoding a detectable marker.

81. The method of claim 80, wherein the detectable marker is a FLAG epitope.

82. The method of claim 72, wherein the eukaryotic cell is a mammalian cell.

83. The method of claim 82, wherein the cell is a human cell.

84. The method of claim 83, wherein the cell is a human 293T HEK cell.

85. The method of claim 82, wherein the eukaryotic cell is in, or is introduced into, a mammal.

86. The method of claim 85, wherein the mammal is a human.

87. The method of claim 86, wherein the human has a mitochondrial disorder.

88. The method of claim 87, wherein the mitochondrial disorder is associated with a mutation in mtDNA.

89. The method of claim 88, wherein the mutation is a point mutation.

90. The method of claim 88, wherein the mitochondrial disorder is selected from the group consisting of FBSN (familial bilateral striatal necrosis), NARP (neuropathy, ataxia, and retinitis pigmentosa), and MILS (maternally-inherited Leigh syndrome)

91. The method of claim 90, wherein the peptide is human ATPase 6 subunit of F₀F₁-ATP synthase.


SEQUENCE LISTING (7 sequences)

SEQ ID NO: 1 - protein, 340 aa, Chlamydomonas reinhardtii
MALQQAAPRVFGLLGRAPVALGQSGILTGSSGFKNQGFNGSLQSVENHVYAQAFSTSSQEEQAAPSIQGASGMKLPGMAGSMLLGKSRSGLRTGSMVPFAAQQAMNMSTAAQVQSGATVNSLLLGIYRFWRSQAPMDKPHAPVDDRMLPAIVDASDNRAALGTWATALFCTILASNLLGLVPTNEAPTSGLGFATGLGVSVWATATTLGLYKLGFSFPGHFIPGGTPWPMAFIFVPLETISYTFRAVSLGVRLWVNMLAGHTLLHILTGMALALPFSLSFFAMVPATFAVACLLSALVGLEYLVAVLQSGVFSILSTVYVGEFNSVKLAGPLAKVVKKAL

SEQ ID NO: 2 - protein, 249 aa, Prototheca wickerhamii
MISTPLEQFTISSIIPLHIGNYYFSFTNSSLYIFLAIGTAGLLFHFVTQNGGFLVPSRWQSLVEMIYEFVRSLIQEQIGAKGRKYFPLVFTLFVFLLFTNLIGMIPYSFTATSHLAVTFGLSLSLFIAITIIGFQVHGLHFFSFLLPKGAPLILAPLLVVLELVSYSFRAISLGVRLFANMMAGHTLVKILSGFAWTMLSVGGILSVASILPFAVVFALIGLEIGVACLQAYVFTILVCIYLNDAINLH

SEQ ID NO: 3 - protein, 259 aa, Saccharomyces cerevisiae
MFNLLNTYITSPLDQFEIRTLFGLQSSFIDLSCLNLTTFSLYTIIVLLVITSLYTLTNNNNKIIGSRWLISQEAIYDTIMNMTKGQIGGKNWGLYFPMIFTLFMFIFIANLISMIPYSFALSAHLVFIISLSIVIWLGNTILGLYKHGWVFFSLFVPAGTPLPLVPLLVIIETLSYFARAISLGLRLGSNILAGHLLMVILAGLTFNFMLINLFTLVFGFVPLAMILAIMMLEFAIGIIQGYVWAILTASYLKDAVYLH

SEQ ID NO: 4 - protein, 226 aa, Homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLHDNT

SEQ ID NO: 5 - protein, 8 aa, Artificial sequence
  MISC_FEATURE (1)..(8): FLAG epitope tag
DYKDDDDK

SEQ ID NO: 6 - DNA, 2222 bp, Chlamydomonas reinhardtii
ttctagacgaagaatatagattggattaaccatggcgctgcagcaggctgcgccgagggtgtttggcctcttggggcgtgcccctgtcgctctgggccagagcggcattctgactggtagcagcggcttcaagaaccagggattcaatggtgaggcggctggccgatttatgtgctgcgaacagctctttcgcggcaccatcacgagctgcgtgcttccggacctgcctcacttgctcggctcccggatatctcaatcccacaggctccctgcagtctgtggagaaccatgtgtatgcccaggtgagctgcggaatgctggtcggtgtcgcgagattgctcctcgctgatgttgggactgactcctgctcgcaggccttcagcacctcttctcaggaggagcaggctgctcccagcatccaggtaggagccgtgctagcgcagaagggccgccgcaaaacttgtcttttcggtatcggagtagagctcgaccgggcgtgtcctggccgcaagggtgcttcgggcataaagctgcctggtatggctggtagcattctgctgggcaagtcgcgcagcggcctgcgcaccggctcgatggtgccattcgctgcccagcaggcgatgaacatgtcgacgggtgagcgccgaggtgcagaacataaaggctacgggctgcattacgggctttagcgtgaaaggattgtggggagctctgggttcgggagtggtcctgagggcggggaaactctggagggtcggcgacctggtgtgggtttgcgtgtaagctgctgccctcactcagctgctgcccccactcctcgcgcgcacgcgatttgcggtctcactccacgcgcccataatgctaccatcgcccttccttgcttgtaactgcagccgcccaggtgcagtcgggcgccaccgtgaactccctgctgctcggcatctaccgcttctggcgcagccaggcgcccatggataagccgcacgcgcccgtggatgaccgcatgctccccgccattgtggatgctagcgacaaccgcgcggccctcggcacgtaagcagctgaggcaatcctgaggcatggtcggggcgagcagggaggaagtcctgagctgcggacaagcctaatgccggggctacggctgctgctggagaggggtgcggattgaggcaattgtgcggttacgggggcattcacctgctgacaatcatgagcgaggaataagcaggatggtgcgttgcattgggacgagaatctatgctgggatccttgacgcggtctacctcctgctcctgcgcctattcccgccccgcagctgggccaccgccctgttctgcaccatcctggcctccaacttgctgggtctggtgcccaccaacgaggcgcccacctccggcctgggcttcgccaccggcctgggtgtgagcgtgtgggccaccgccaccaccctgggtctgtacaagctgggcttctccttccccgggcacttcatccctggtaggaacgggcgggtgcgggagccaacgcattgaaggttgtggagcagcgggtttgtgtttgcacaggtcagcaccgcgtgcggggtgtgccgggtatgcacggcatgtgctgggtgcgtgcacgccaacaccgaatggcaaaacttgtgcctgactagttggttgttgtccttgccgaccgcaggcggcaccccccggcccatggccttcatcttcgtgcccctggagaccatttcgtacaccttccgtgctgtttccctgggtgtccgtctgtgggtcaacatgcttgccggacacacgctgctgcacatcctgacgggtgagtcgctgccgttcgcacagctgcactgtaaactacagattaggtgcgctctctgggaccctgcgacgccacacctacaccgcctgctttccagaacccacctggctgccaccccgcctccccacgccgtctctctcaaccggtactcttccttgcaggcatggcgctggcgctgcccttctcgctgtcgttcttcgccatggtgcccgccaccttcgccgtcgcctgcctgctgtcggcgctggtgggcctggagtacctggtggcggtgctgcagtcgggtgtgttctccatcctgtccaccgtctacgttggcgagttcaacagcgtcaagctggcgggcccgctggctaaggtggtcaagaaggcgctgtaagcagcctcggaatgggacggtgtga

SEQ ID NO: 7 - DNA, 1079 bp, Chlamydomonas reinhardtii
ttctagacgaagaatatagattggattaaccatggcgctgcagcaggctgcgccgagggtgttcggcctcttggggcgtgcccccgtcgctctgggccagagcggcattctgactggtagcagcggcttcaagaaccagggattcaacggctccctgcagtctgtggagaaccatgtgtatgcccaggccttcagcacctcttctcaggaggagcaggctgctcccagcattcagggtgcttcgggcatgaagctgcctggtatggctggtagcatgctgcttggcaagtcgcgcagcggcctgcgcaccggctcgatggtgccattcgctgcccagcaggcgatgaacatgtcgacggccgcccaggtgcagtcgggcgccaccgtgaactccctgctgctcggcatctaccgcttctggcgcagccaggcgcccatggataagccgcacgcgcccgtggatgaccgcatgctccccgccattgtggatgctagcgacaactgcgcggccctgggaacctgggccaccgccctgttctgcaccatcctggcctccaacttgctgggtctggtgcccaccaacgaggcgcccacctccggcctgggcttcgccaccggcctgggtgtgagcgtgtgggtcaccgccaccaccctgggtctgtacaagctgggcttctccttccccgggcacttcatccctggcggcaccccctggcccatggccttcatcttcgtgcccctggagaccatttcgtacaccttccgtgctgtttccctgggtgtccgtctgtgggtcaacatgctggccggacacacgctgctgcacattctgacgggcatggcgctggcgctgcctttctcgctgtcgttcttcgccatggtgcccgccaccttcgccgtcgcctgcctgctgtcggcgctggtgggcctggagtacctggtggcggtgctgcagtcgggtgtgttctccatcctgtccaccgtctacgttggcgagttcaacagcgtcaagctggcgggcccgctggctaaggtggtcaagaaggcgctgtaagcggcctcggaatgggacggtgtga